(19) United States
(12) Patent Application Publication
(10) Pub. No.: US 2014/0162250 A1
(43) Pub. Date: Jun. 12, 2014

(54) MARKER-ASSISTED SELECTION OF TOLERANCE TO CHLORIDE SALT STRESS

(71) Applicant: PIONEER HI-BRED INTERNATIONAL, INC., Johnston, IA (US)

(72) Inventors: Mark J. Hood, Memphis, TN (US); David L. Hyten, Johnston, IA (US); Andrea B. Kalvig, Waukee, IA (US); Jason C. Raines, Crawfordsville, AR (US); Joshua M. Shendelman, Ankeny, IA (US); John B. Woodward, Ankeny, IA (US); Yanwen Xiong, Johnston, IA (US)

(73) Assignee: Pioneer Hi-Bred International, Inc., Johnston, IA (US)

(21) Appl. No.: 13/782,044

(22) Filed: Mar. 1, 2013

Related U.S. Application Data
(60) Provisional application No. 61/736,268, filed on Dec. 12, 2012.

(51) Int. Cl. C12Q 1/68

(52) U.S. Cl. CPC C12Q 1/6895
USPC 435/6.11; 536/24.3

(57) ABSTRACT

Various methods and compositions are provided for identifying and/or selecting soybean plants or soybean germplasm with tolerance to chloride salt stress. In certain embodiments, the method comprises detecting at least one marker locus that is associated with tolerance to chloride salt stress. In other embodiments, the method further comprises detecting at least one marker profile or haplotype associated with chloride salt stress tolerance. In further embodiments, the method comprises crossing a selected soybean plant with a second soybean plant. Further provided are markers, primers, probes and kits useful for identifying and/or selecting soybean plants or soybean germplasm with tolerance to chloride salt stress.

### CROSS-REFERENCE TO RELATED APPLICATIONS

This application claims the benefit of U.S. Provisional Application No. 61/736, 268, filed Dec. 12, 2012, which is hereby incorporated herein in its entirety by reference.

### FIELD OF THE INVENTION

This invention relates to methods of identifying and/or selecting soybean plants or germplasm that display tolerance to chloride salt stress.

### REFERENCE TO A SEQUENCE LISTING SUBMITTED AS A TEXT FILE VIA EFS-WEB

The official copy of the sequence listing is submitted concurrently with the specification as a text file via EFS-Web, in compliance with the American Standard Code for Information Interchange (ASCII), with a file name of 430053seqlist.txt, a creation date of Feb. 19, 2013 and a size of 17 KB. The sequence listing filed via EFS-Web is part of the specification and is hereby incorporated in its entirety by reference herein.

### BACKGROUND

Soybeans (Glycine max L. Merr.) are a major cash crop and investment commodity in North America and elsewhere. Soybean oil is one of the most widely used edible oils, and soybeans are used worldwide both in animal feed and in human food production. Additionally, soybean utilization is expanding to industrial, manufacturing, and pharmaceutical applications.

Molecular markers have been used to selectively improve soybean crops through the use of marker assisted selection. Any detectible polymorphic trait can be used as a marker so long as it is inherited differentially and exhibits linkage disequilibrium with a phenotypic trait of interest. A number of soybean markers have been mapped and linkage groups created, as described in Cregan, et al., “An Integrated Genetic Linkage Map of the Soybean Genome” (1999) Crop Science 39:1464-90, Choi, et al., “A Soybean Transcript Map: Gene Distribution, Haplotype and Single-Nucleotide Polymorphism Analysis” (2007) Genetics 176:685-96, and Hyten, et al. “A High Density Integrated Genetic Linkage Map of Soybean and the Development of a 1536 Universal Soy Linkage Panel for Quantitative Trait Locus Mapping” (2010) Crop Science 50:960-968. Many soybean markers are publicly available at the USDA affiliated soybase website (www.soybase.org).

High chloride salt concentrations in soils are a major abiotic stress factor affecting soybean. Chloride salt stress occurs in multiple soybean production areas across the United States. In soybean, salt stress inhibits seed germination and plant growth, reduces root nodule formation and decreases yield. Field testing for chloride salt stress tolerance is laborious, expensive and challenging, which has delayed the widespread development of tolerant lines.

There remains a need for soybean plants with tolerance to chloride salt stress and methods for identifying and selecting such plants.

### SUMMARY

Various methods and compositions are provided for identifying and/or selecting soybean plants or soybean germplasm with tolerance to chloride salt stress. In certain embodiments, the method comprises detecting at least one marker locus that is associated with tolerance to chloride salt stress. In other embodiments, the method further comprises detecting at least one marker profile or haplotype associated with chloride salt stress tolerance. In further embodiments, the method comprises crossing a selected soybean plant with a second soybean plant. Further provided are markers, primers, probes and kits useful for identifying and/or selecting soybean plants or soybean germplasm with tolerance to chloride salt stress.

### BRIEF DESCRIPTION OF THE FIGURES

FIG. 1A-D depicts non-limiting examples of marker loci located within, linked to, or closely linked to the genomic regions or intervals provided herein.

### DETAILED DESCRIPTION

Before describing the present invention in detail, it is to be understood that this invention is not limited to particular embodiments, which can, of course, vary. It is also to be understood that the terminology used herein is for the purpose of describing particular embodiments only, and is not intended to be limiting.

Certain definitions used in the specification and claims are provided below. In order to provide a clear and consistent understanding of the specification and claims, including the scope to be given such terms, the following definitions are provided:

As used in this specification and the appended claims, terms in the singular and the singular forms “a,” “an,” and “the,” for example, include plural referents unless the content clearly dictates otherwise. Thus, for example, reference to “plant,” “the plant,” or “a plant” also includes a plurality of plants; also, depending on the context, use of the term “plant” can also include genetically similar or identical progeny of that plant; use of the term “a nucleic acid” optionally includes, as a practical matter, many copies of that nucleic acid molecule; similarly, the term “probe” optionally (and typically) encompasses many similar or identical probe molecules.

Additionally, as used herein, “comprising” is to be interpreted as specifying the presence of the stated features, integers, steps, or components as referred to, but does not preclude the presence or addition of one or more features, integers, steps, or components, or groups thereof. Thus, for example, a kit comprising one pair of oligonucleotide primers may have two or more pairs of oligonucleotide primers. Additionally, the term “comprising” is intended to include examples encompassed by the terms “consisting essentially of” and “consisting of.” Similarly, the term “consisting essentially of” is intended to include examples encompassed by the term “consisting of.”

“Agronomics,” “agronomic traits,” and “agronomic performance” refer to the traits (and underlying genetic elements) of a given plant variety that contribute to yield over the course of a growing season. Individual agronomic traits include emergence vigor, vegetative vigor, stress tolerance, disease resistance or tolerance, insect resistance or tolerance, herbicide resistance, branching, flowering, seed set, seed size, seed density, standability, threshability, and the like.

“Allele” means any of one or more alternative forms of a genetic sequence. In a diploid cell or organism, the two alleles of a given sequence typically occupy corresponding loci on a pair of homologous chromosomes. With regard to a SNP marker, allele refers to the specific nucleotide base present at that SNP locus in that individual plant.

The term “amplifying” in the context of nucleic acid amplification is any process whereby additional copies of a selected nucleic acid (or a transcribed form thereof) are produced. Typical amplification methods include various polymerase based replication methods, including the polymerase chain reaction (PCR), ligase mediated methods, such as the ligase chain reaction (LCR), and RNA polymerase based amplification (e.g., by transcription) methods. An “amplicon” is an amplified nucleic acid, e.g., a nucleic acid that is produced by amplifying a template nucleic acid by any available amplification method (e.g., PCR, LCR, transcription, or the like).

An “ancestral line” is a parent line used as a source of genes, e.g., for the development of elite lines.

An “ancestral population” is a group of ancestors that have contributed the bulk of the genetic variation that was used to develop elite lines.

“Backcrossing” is a process in which a breeder crosses a progeny variety back to one of the parental genotypes one or more times.

The term “chromosome segment” designates a contiguous linear span of genomic DNA that resides in planta on a single chromosome.

Chloride field score is a visual score from 1 to 9 comparing all genotypes in a given test. The score is based on the extent and distribution of chlorotic symptoms in the leaves. Mild symptoms include faint chlorosis between leaf veins. As symptoms increase, the chlorosis becomes more severe, including impact to leaf margins. In the most severe cases, leaf tissue will die. A score of 1 indicates severe symptoms of leaf yellowing and necrosis. Increasing visual scores from 2 to 8 indicate additional levels of tolerance, while a score of 9 indicates no symptoms.

“Cultivar” and “variety” are used synonymously and mean a group of plants within a species (e.g., Glycine max) that share certain genetic traits that separate them from other possible varieties within that species. Soybean cultivars are typically inbred lines produced after several generations of self-pollination, however hybrid varieties may also be produced. Both inbred or hybrid varieties may be developing in a breeding program using doubled haploid technology. Individuals within a soybean cultivar are homogeneous, nearly genetically identical, with most loci in the homozygous state.

An “elite line” is an agronomically superior line that has resulted from many cycles of breeding and selection for superior agronomic performance. Numerous elite lines are available and known to those of skill in the art of soybean breeding.

An “elite population” is an assortment of elite individuals or lines that can be used to represent the state of the art in terms of agronomically superior genotypes of a given crop species, such as soybean.

An “exotic soybean strain” or an “exotic soybean germplasm” is a strain or germplasm derived from a soybean not belonging to an available elite soybean line or strain of germplasm. In the context of a cross between two soybean plants or strains of germplasm, an exotic germplasm is not closely related by descent to the elite germplasm with which it is crossed. Most commonly, the exotic germplasm is not derived from any known elite line of soybean, but rather is selected to introduce novel genetic elements (typically novel alleles) into a breeding program.

A “genetic map” is a description of genetic linkage relationships among loci on one or more chromosomes (or linkage groups) within a given species, generally depicted in a diagrammatic or tabular form.

“Genotype” refers to the genetic constitution of a cell or organism.

“Germplasm” means the genetic material that comprises the physical foundation of the hereditary qualities of an organism. As used herein, germplasm includes seeds and living tissue from which new plants may be grown; or, another plant part, such as leaf, stem, pollen, or cells, that may be cultured into a whole plant. Germplasm resources provide sources of genetic traits used by plant breeders to improve commercial cultivars.

An individual is “homozygous” if the individual has only one type of allele at a given locus (e.g., a diploid individual has a copy of the same allele at a locus for each of two homologous chromosomes). An individual is “heterozygous” if more than one allele type is present at a given locus (e.g., a diploid individual with one copy each of two different alleles). The term “homogeneity” indicates that members of a group have the same genotype at one or more specific loci. In contrast, the term “heterogeneity” is used to indicate that individuals within the group differ in genotype at one or more specific loci.

“Introgression” means the entry or introduction of a gene, QTL, haplotype, marker profile, trait, or trait locus from the genome of one plant into the genome of another plant.

The terms “label” or “detectable label” refer to a molecule capable of detection. A detectable label can also include a combination of a reporter and a quencher, such as are employed in FRET probes or TaqMan™ probes. The term “reporter” refers to a substance or a portion thereof which is capable of exhibiting a detectable signal, which signal can be suppressed by a quencher. The detectable signal of the reporter is, e.g., fluorescence in the detectable range. The term “quencher” refers to a substance or portion thereof which is capable of suppressing, reducing, inhibiting, etc., the detectable signal produced by the reporter. As used herein, the terms “quenching” and “fluorescence energy transfer” refer to the process whereby, when a reporter and a quencher are in close proximity, and the reporter is excited by an energy source, a substantial portion of the energy of the excited state non-radiatively transfers to the quencher where it either dissipates non-radiatively or is emitted at a different emission wavelength than that of the reporter.

A “line” or “strain” is a group of individuals of identical parentage that are generally inbred to some degree and that are generally homozygous and homogeneous at most loci (isogenic or near isogenic). A “subline” refers to an inbred subset of descendants that are genetically distinct from other similarly inbred subsets descended from the same progenitor. Traditionally, a subline has been derived by inbreeding the seed from an individual soybean plant selected at the F3 to F5 generation until the residual segregating loci are “fixed” or homozygous across most or all loci. Commercial soybean varieties (or lines) are typically produced by aggregating (“bulking”) the self-pollinated progeny of a single F3 to F5 plant from a controlled cross between 2 genetically different parents. While the variety typically appears uniform, the self-pollinating variety derived from the selected plant eventually (e.g., F8) becomes a mixture of homozygous plants that can vary in genotype at any locus that was heterozygous in the originally selected F3 to F5 plant. Marker-based sublines that differ from each other based on qualitative polymorphism at the DNA level at one or more specific marker loci are derived by genotyping a sample of seed derived from individual self-pollinated progeny derived from a selected F3-F5 plant. The seed sample can be genotyped directly as seed, or as plant tissue grown from such a seed sample. Optionally, seed sharing a common genotype at the specified locus (or loci) are bulked providing a subline that is genetically homogenous at identified loci important for a trait of interest (e.g., yield, tolerance, etc.).

“Linkage” refers to the tendency for alleles to segregate together more often than expected by chance if their transmission was independent. Typically, linkage refers to loci on the same chromosome. Genetic recombination occurs with an assumed random frequency over the entire genome. Genetic maps are constructed by measuring the frequency of recombination between pairs of traits or markers, the lower the frequency of recombination, the greater the degree of linkage.

“Linkage disequilibrium” refers to a non-random association of alleles within a population.

“Linkage group” (LG) refers to traits or markers that co-segregate. A linkage group generally corresponds to a chromosomal region containing genetic material that encodes the traits or markers.

“Locus” is a defined segment of DNA.

A “map location” or “map position” is an assigned location on a genetic map relative to linked genetic markers where a specified marker can be found within a given species. Map positions are generally provided in centimorgans (cM). A “physical position” or “physical location” or “physical map location” is the position, typically in nucleotides bases, of a particular nucleotide, such as a SNP nucleotide, on a chromosome.

“Mapping” is the process of defining linkage or association among loci through the use of markers segregating within populations. Linkage mapping relies on the standard genetic principles of recombination frequency among loci and identifying linkage, while association mapping relies on linkage disequilibrium among loci.

“Marker” or “molecular marker” or “marker locus” is a term used to denote a nucleic acid or amino acid sequence that is sufficiently unique to characterize a specific locus on the genome. Any detectable polymorphic trait can be used as a marker so long as it is inherited differentially and exhibits linkage disequilibrium with a phenotypic trait of interest.

“Marker assisted selection” refers to the process of selecting a desired trait or traits in a plant or plants by detecting one or more nucleic acids from the plant, where the nucleic acid is associated with or linked to the desired trait, and then selecting the plant or germplasm possessing those one or more nucleic acids.

“Haplotype” refers to a combination of particular alleles present within a particular plant's genome at two or more linked marker loci, for instance at two or more loci on a particular linkage group. For instance, in one example, two specific marker loci on LG-N are used to define a haplotype for a particular plant. In still further examples, 3, 4, 5, 6, 7, 8, 9, 10, 11, 12, 13, 14, 15, 16, 17, 18, 19, 20, or more linked marker loci are used to define a haplotype for a particular plant.

In certain examples, multiple marker loci or haplotypes are used to define a “marker profile”. As used herein, “marker profile” means the combination of two or more marker loci, haplotypes, or any combination thereof, within a particular plant's genome. For instance, in one example, a particular combination of marker loci or a particular combination of haplotypes define the marker profile of a particular plant.

The term “plant” includes reference to an immature or mature whole plant, including a plant from which seed or grain or anthers have been removed. Seed or embryo that will produce the plant is also considered to be the plant.

“Plant parts” means any portion or piece of a plant, including leaves, stems, buds, roots, root tips, anthers, seed, grain, embryo, pollen, ovules, flowers, cotyledons, hypocotyls, pods, flowers, shoots, stalks, tissues, tissue cultures, cells and the like.

“Polymorphism” means a change or difference between two related nucleic acids. A “nucleotide polymorphism” refers to a nucleotide that is different in one sequence when compared to a related sequence when the two nucleic acids are aligned for maximal correspondence.

“Polynucleotide,” “polynucleotide sequence,” “nucleic acid,” “nucleic acid molecule,” “nucleic acid sequence,” “nucleic acid fragment,” and “oligonucleotide” are used interchangeably hereinto indicate a polymer of nucleotides that is single- or multi-stranded, that optionally contains synthetic, non-natural, or altered RNA or DNA nucleotide bases. A DNA polynucleotide may be comprised of one or more strands of cDNA, genomic DNA, synthetic DNA, or mixtures thereof.

“Primer” refers to an oligonucleotide which is capable of acting as a point of initiation of nucleic acid synthesis or replication along a complementary strand when placed under conditions in which synthesis of a complementary strand is catalyzed by a polymerase. Typically, primers are about 10 to 30 nucleotides in length, but longer or shorter sequences can be employed. Primers may be provided in double-stranded form, though the single-stranded form is more typically used. A primer can further contain a detectable label, for example a 5′ end label.

“Probe” refers to an oligonucleotide that is complementary (though not necessarily fully complementary) to a polynucleotide of interest and forms a duplexed structure by hybridization with at least one strand of the polynucleotide of interest. Typically, probes are oligonucleotides from 10 to 50 nucleotides in length, but longer or shorter sequences can be employed. A probe can further contain a detectable label.

“Quantitative trait loci” or “QTL” refer to the genetic elements controlling a quantitative trait.

“Recombination frequency” is the frequency of a crossing over event (recombination) between two genetic loci. Recombination frequency can be observed by following the segregation of markers and/or traits during meiosis.

“Tolerance and “improved tolerance” are used interchangeably herein and refer to any type of increase in resistance or tolerance to, or any type of decrease in susceptibility.

A “tolerant plant” or “tolerant plant variety” need not possess absolute or complete tolerance. Instead, a “tolerant plant,” “tolerant plant variety,” or a plant or plant variety with “improved tolerance” will have a level of resistance or tolerance which is higher than that of a comparable susceptible plant or variety.

“Self-crossing” or “self-pollination” or “selfing” is a process through which a breeder crosses a plant with itself; for example, a second generation hybrid F2 with itself to yield progeny designated F2:3.

“SNP” or “single nucleotide polymorphism” means a sequence variation that occurs when a single nucleotide (A, T, C, or G) in the genome sequence is altered or variable. “SNP markers” exist when SNPs are mapped to sites on the soybean genome.

The term “yield” refers to the productivity per unit area of a particular plant product of commercial value. For example, yield of soybean is commonly measured in bushels of seed per acre or metric tons of seed per hectare per season. Yield is affected by both genetic and environmental factors.

As used herein, an “isolated” or “purified” polynucleotide or polypeptide, or biologically active portion thereof, is substantially or essentially free from components that normally accompany or interact with the polynucleotide or polypeptide as found in its naturally occurring environment. Thus, an isolated or purified polynucleotide or polypeptide is substantially free of other cellular material or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various embodiments, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. A polypeptide that is substantially free of cellular material includes preparations of polypeptides having less than about 30%, 20%, 10%, 5%, or 1% (by dry weight) of contaminating protein. When the polypeptide or biologically active portion thereof is recombinantly produced, culture medium typically represents less than about 30%, 20%, 10%, 5%, or 1% (by dry weight) of chemical precursors or non-protein-of-interest chemicals.

Standard recombinant DNA and molecular cloning techniques used herein are well known in the art and are described more fully in Sambrook, J., Fritsch, E. F. and Maniatis, T. Molecular Cloning: A Laboratory Manual; Cold Spring Harbor Laboratory Press: Cold Spring Harbor, 1989 (hereinafter “Sambrook”).

Saline soils and water in areas used for agricultural production can limit crop production due to both sodium and chloride toxicity. Since plants take up and transport sodium and chloride differently, and have different mechanisms for dealing with the toxicity of each. Each has separate effects, for example sodium can interfere with potassium or inactivate enzymes, which chloride can disrupt photosynthesis. Chloride can come from various sources including the soil, from irrigation water, and/or from fertilizers, such as muriate of potash (potassium chloride). Other fertilizers or water sources may be used having lower chloride, but may be more expensive. One known mechanism for chloride tolerance is to limit transport of chloride to the leaves and stems, these plants are called excluders and store the chloride in the roots. The other class are known as includers, in these varieties chloride is taken in and transported to the top of the plant (leaves and stems), and at high chloride concentrations toxicity symptoms may occur.

Methods are provided for identifying and/or selecting a soybean plant or soybean germplasm that displays tolerance to chloride salt stress. The method comprises detecting in the soybean plant or germplasm, or a part thereof, at least one marker locus associated with tolerance to chloride salt stress. Also provided are isolated polynucleotides and kits for use in identifying and/or detecting a soybean plant or soybean germplasm that displays tolerance to chloride salt stress.

Provided herein, marker loci associated with soybean chloride salt stress tolerance have been identified and fine mapped to a genomic region on linkage group N. This region on linkage group N comprises a known Quantitative Trait Locus (QTL) for chloride salt stress tolerance. The known QTL, which maps between markers Sat_{—}091 and Satt237, was characterized by Lee et al. ((2004) “A major QTL conditioning salt tolerance in S-100 soybean and descendent cultivars” Theor. Appl. Genet. 109:1610-19). Herein, this region has been further characterized, and it was discovered that the QTL extends to include the region between the S04733-1-A and S16227-001-K001 markers on linkage group N.

Marker loci, haplotypes and marker profiles associated with soybean tolerance to chloride salt stress, are provided. Further provided are genomic regions that represent QTLs which are associated with soybean tolerance to chloride salt stress. These results have important implications for soybean production, as identifying markers that can be used for selection of chloride salt stress tolerance will greatly expedite the development of chloride salt stress tolerance into elite cultivars.

In certain embodiments, soybean plants or germplasm are identified that have at least one favorable allele, marker locus, haplotype, or marker profile that positively correlates with tolerance or improved tolerance to chloride salt stress. However, in other embodiments, it is useful for exclusionary purposes during breeding to identify alleles, marker loci, haplotypes, or marker profiles that negatively correlate with tolerance, for example, to eliminate such plants or germplasm from subsequent rounds of breeding.

In one embodiment, marker loci useful for identifying a first soybean plant or first soybean germplasm that displays tolerance to chloride salt stress are localized to a genomic region between about position 40454221 and about position 40759329 on linkage group N (G. max chromosome 3) based on the Glyma1 Williams82 soybean reference assembly (Schmutz et al. (2010) “Genome sequence of the palaeopolyploid soybean.” Nature 463:178-183; and www.phytozome.net/soybean). In a specific embodiment, the marker locus comprises one or more of GM03:40563114, GM03:40576895, GM03:40489573, GM03:40489574, GM03:40557669, GM03:40591130, GM03:40703866, GM03:40554209, GM03:40589164, GM03:40606905, GM03:40632077, GM03:40705541, GM03:40576921, S06578-1-A, S16256-001-Q001, S16255-001-Q001, S16254-001-Q001, S16253-001-Q001, S16252-001-Q001, S16232-001-Q001 or a marker closely linked thereto.

In other embodiments, marker loci useful for identifying a first soybean plant or first soybean germplasm that display tolerance to chloride salt stress are localized to a genomic region between about marker S04733-1-A and about marker S16227-001-K001 on linkage group N. In a specific embodiment, the marker locus comprises one or more of S04733-1-A, S12869-1-Q1, S00145-1-A, S16226-001-K001, S16227-001-K001 or a marker closely linked thereto.

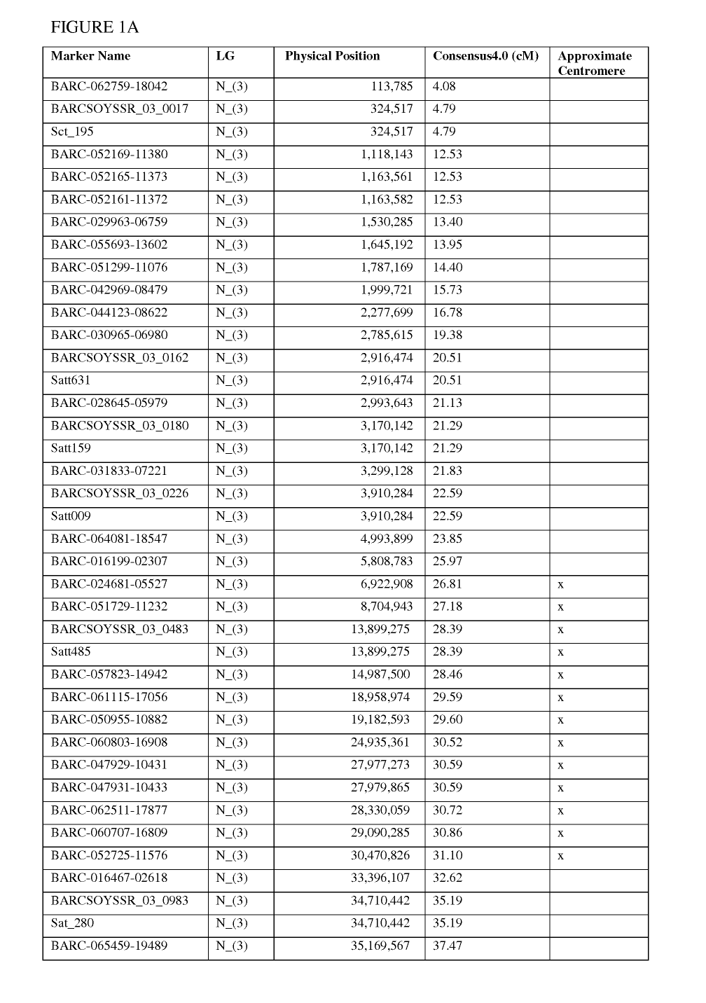

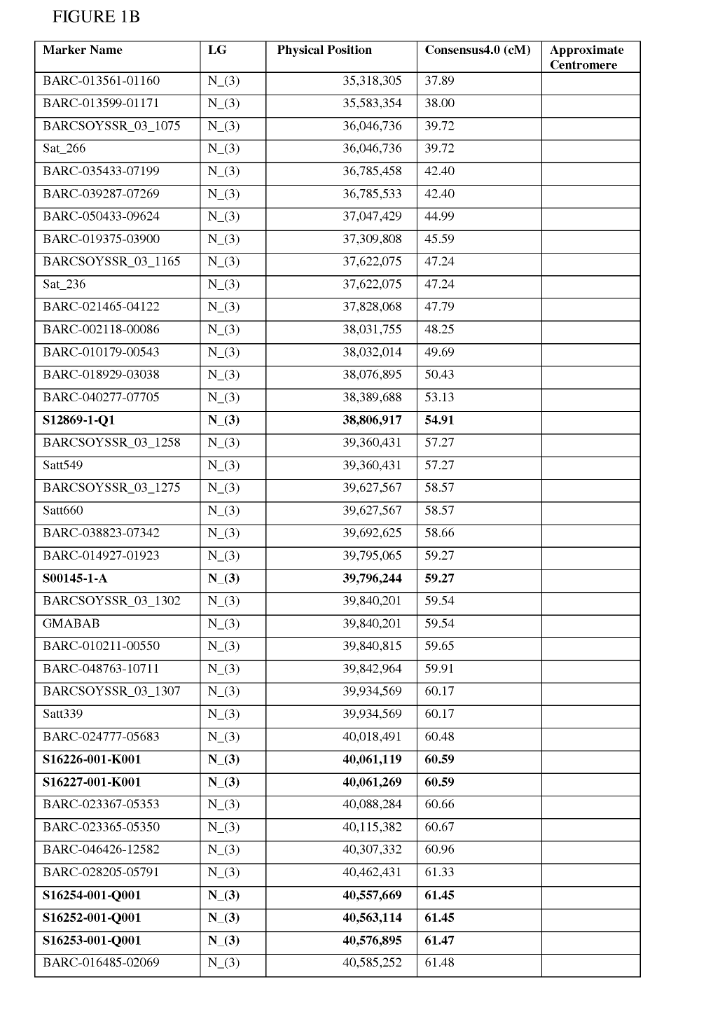

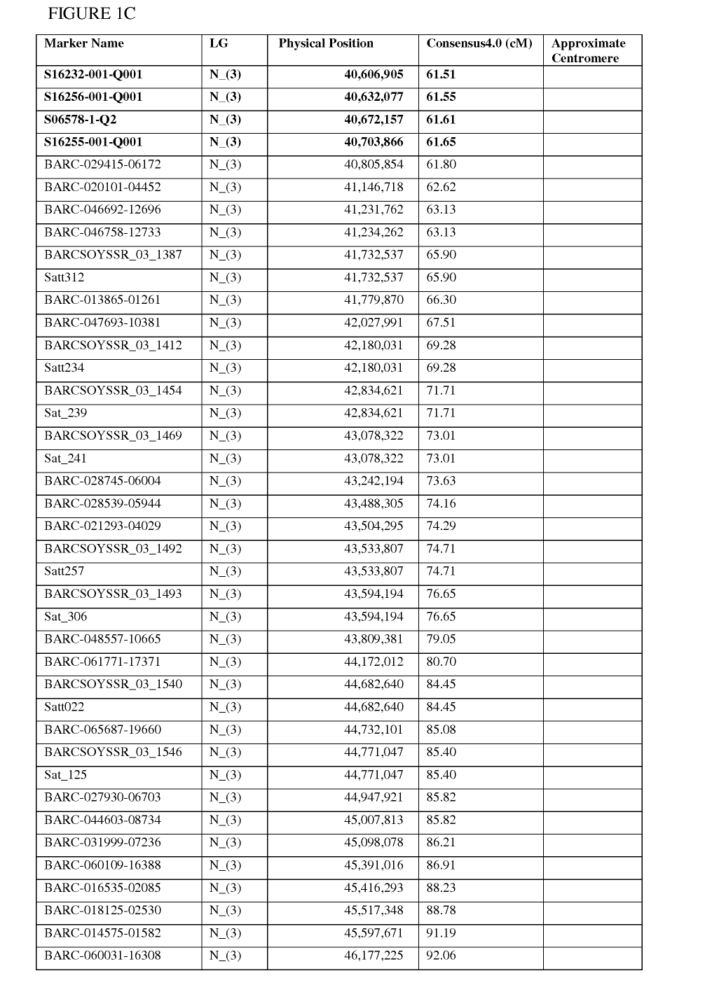

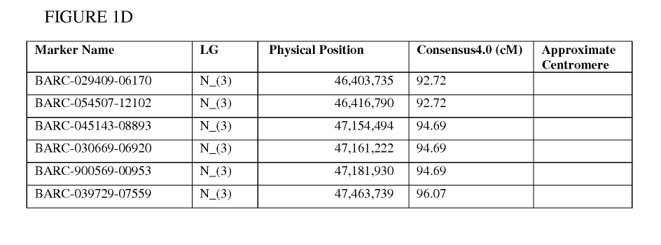

Non-limiting examples of marker loci located within, linked to, or closely linked to these genomic regions or intervals are illustrated in FIG. 1 and are listed in Tables 1-9.

In certain embodiments, multiple marker loci that collectively make up the chloride salt stress tolerance haplotype of interest are investigated. For example, 2, 3, 4, 5, 6, 7, 8, 9, 10, 11, 12, 13, 14, 15, 16, 17, 18, 19, 20, or more of the various marker loci provided herein can comprise a chloride salt stress tolerance haplotype. In some embodiments, the haplotype comprises two or more of any combination of the following marker loci: (a) any marker loci found between position 40454221 and 40759329 on linkage group N; (b) marker loci comprising GM03:40563114, GM03:40576895, GM03:40489573, GM03:40489574, GM03:40557669, GM03:40591130, GM03:40703866, GM03:40554209, GM03:40589164, GM03:40606905, GM03:40632077, GM03:40705541, GM03:40576921, S06578-1-A, S16256-001-Q001, S16255-001-Q001, S16254-001-Q001, S16253-001-Q001, S16252-001-Q001, S16232-001-Q001, or a closely linked marker; or (c) any marker loci between about marker S04733-1-A and about marker S16227-001-K001 on linkage group N; and/or (d) marker loci comprising S04733-1-A, S12869-1-Q1, S00145-1-A, S16226-001-K001, S16227-001-K001 or a closely linked marker.

In a specific embodiment, the haplotype can comprise two or more of the marker loci found between position 40454221 and 40759329 on linkage group N, including GM03:40563114, GM03:40576895, GM03:40489573, GM03:40489574, GM03:40557669, GM03:40591130, GM03:40703866, GM03:40554209, GM03:40589164, GM03:40606905, GM03:40632077, GM03:40705541, GM03:40576921, S06578-1-A, S16256-001-Q001, S16255-001-Q001, S16254-001-Q001, S16253-001-Q001, S16252-001-Q001 or S16232-001-Q001.

In certain embodiments, two or more marker loci or haplotypes can collectively make up a marker profile. The marker profile can comprise any two or more marker loci: (a) marker loci found between position 40454221 and 40759329 on linkage group N; (b) marker loci comprising GM03:40563114, GM03:40576895, GM03:40489573, GM03:40489574, GM03:40557669, GM03:40591130, GM03:40703866, GM03:40554209, GM03:40589164, GM03:40606905, GM03:40632077, GM03:40705541, GM03:40576921, S06578-1-A, S16256-001-Q001, S16255-001-Q001, S16254-001-Q001, S16253-001-Q001, S16252-001-Q001, S16232-001-Q001 or a closely linked marker; (c) any marker loci between about marker S04733-1-A and about marker S16227-001-K001 on linkage group N; and/or (d) marker loci comprising S04733-1-A, S12869-1-Q1, S00145-1-A, S16226-001-K001 or S16227-001-K001, or a closely linked marker. Any of the marker loci in any of the genomic regions disclosed herein can be combined in the marker profile. For example, the marker profile can comprise 2, 3, 4, 5, 6, 7, 8, 9, 10, 11, 12, 13, 14, 15, 16, 17, 18, 19, 20, or more marker loci or haplotypes associated with chloride salt stress tolerance provided herein.

Not only can one detect the various markers provided herein, it is recognized that one could detect any markers that are closely linked to the various markers discussed herein. In addition to the markers discussed herein, information regarding useful soybean markers can be found, for example, on the USDA's Soybase website, available at www.soybase.org. One of skill in the art will recognize that the identification of favorable marker alleles may be germplasm-specific. The determination of which marker alleles correlate with tolerance (or susceptibility) is determined for the particular germplasm under study. One of skill will also recognize that methods for identifying the favorable alleles are routine and well known in the art, and furthermore, that the identification and use of such favorable alleles is well within the scope of the invention.

Various methods are provided to identify soybean plants and/or germplasm with tolerance to chloride salt stress. In one embodiment, the method of identifying comprises detecting at least one marker locus associated with tolerance to chloride salt stress. The term “associated with” in connection with a relationship between a marker locus and a phenotype refers to a statistically significant dependence of marker frequency with respect to a quantitative scale or qualitative gradation of the phenotype. Thus, an allele of a marker is associated with a trait of interest when the allele of the marker locus and the trait phenotypes are found together in the progeny of an organism more often than if the marker genotypes and trait phenotypes segregated separately.

Any combination of the marker loci provided herein can be used in the methods to identify a soybean plant or soybean germplasm that displays tolerance to chloride salt stress. In non-limiting embodiments, the marker loci used to identify a soybean plant or soybean germplasm that displays tolerance to chloride salt stress comprises one or more of GM03:40563114, GM03:40576895, GM03:40489573, GM03:40489574, GM03:40557669, GM03:40591130, GM03:40703866, GM03:40554209, GM03:40589164, GM03:40606905, GM03:40632077, GM03:40705541, GM03:40576921, S06578-1-A, S16256-001-Q001, S16255-001-Q001, S16254-001-Q001, S16253-001-Q001, S16252-001-Q001, S16232-001-Q001 or a closely linked marker. In other non-limiting embodiments, the soybean marker locus comprises at least one of S04733-1-A, S12869-1-Q1, S00145-1-A, S16226-001-K001, S16227-001-K001, or a closely linked marker. Additional marker loci that can be used in the methods provided herein are set forth in FIG. 1 and in Tables 4-9. Thus, any one marker locus or any combination of the markers set forth in FIG. 1 or in Tables 4-9 can be used to aid in identifying and selecting soybean plants or soybean germplasm with tolerance to chloride salt stress.

In one embodiment, a method of identifying a first soybean plant or a first soybean germplasm that displays tolerance to chloride salt stress is provided. The method comprises detecting in the genome of the first soybean plant or first soybean germplasm at least one marker locus that is associated with tolerance. In such a method, the at least one marker locus: (a) can be localized in a genomic region between about position 40454221 and about position 40759329 on linkage group N; (b) can comprise one or more of the marker loci GM03:40563114, GM03:40576895, GM03:40489573, GM03:40489574, GM03:40557669, GM03:40591130, GM03:40703866, GM03:40554209, GM03:40589164, GM03:40606905, GM03:40632077, GM03:40705541, GM03:40576921, S06578-1-A, S16256-001-Q001, S16255-001-Q001, S16254-001-Q001, S16253-001-Q001, S16252-001-Q001, S16232-001-Q001 or a closely linked marker located on linkage group N; and/or (c) can be between about marker S04733-1-A and about marker S16227-001-K001 on linkage group N, including, for example, the marker loci S04733-1-A, S12869-1-Q1, S00145-1-A, S16226-001-K001, S16227-001-K001 or a marker closely linked thereto.

In other embodiments, two or more marker loci are detected in the method. In a specific embodiment, the germplasm is a soybean variety.

In other embodiments, the method further comprises crossing the selected first soybean plant or first soybean germplasm comprising at least one marker locus associated with chloride salt stress tolerance with a second soybean plant or second soybean germplasm. In a further embodiment of the method, the second soybean plant or second soybean germplasm comprises an exotic soybean strain or an elite soybean strain. In some examples the method further comprises producing a progeny, wherein the progeny has improved tolerance to chloride salt stress as compared to a susceptible variety.

In one embodiment, the method of detecting comprises DNA sequencing of at least one of the marker loci provided herein. As used herein, “sequencing” refers to sequencing methods for determining the order of nucleotides in a molecule of DNA. Any sequencing method known in the art can be used in the methods provided herein. Examples of such sequencing methods are provided elsewhere herein.

In another embodiment, the detection method comprises amplifying at least one marker locus and detecting the resulting amplified marker amplicon. In such a method, amplifying comprises (a) admixing an amplification primer or amplification primer pair for each marker locus being amplified with a nucleic acid isolated from the first soybean plant or the first soybean germplasm such that the primer or primer pair is complementary or partially complementary to a variant or fragment of the genomic region comprising the marker locus and is capable of initiating DNA polymerization by a DNA polymerase using the soybean nucleic acid as a template; and (b) extending the primer or primer pair in a DNA polymerization reaction comprising a DNA polymerase and a template nucleic acid to generate at least one amplicon. In such a method, the primer or primer pair can comprise a variant or fragment of one or more of the genomic regions provided herein. In a further embodiment, the method involves amplifying a variant or fragment of one or more polynucleotides comprising SEQ ID NOS: 47, 48, 49, 50, 51, 52, 53, 54, 55, 56, 57, 58 or complements thereof. In one embodiment, the primer or primer pair can comprise at least a portion of one or more polynucleotides comprising SEQ ID NOS: 47, 48, 49, 50, 51, 52, 53, 54, 55, 56, 57, 58 or variants or fragments thereof. In specific embodiments, the primer or primer pair comprises a nucleic acid sequence comprising SEQ ID NOS: 1, 2, 3, 4, 5, 6, 7, 8, 9, 10, 11, 12, 13, 14, 15, 16, 17, 18, 19, 20, 21, 22 or variants or fragments thereof.

In a further embodiment, the method further comprises providing one or more labeled nucleic acid probes suitable for detection of each marker locus being amplified. In such a method, the labeled nucleic acid probe can comprise a sequence comprising a variant or fragment of one or more of the genomic regions provided herein. In one embodiment, the labeled nucleic acid probe can comprise a sequence comprising a variant or fragment of one or more polynucleotides comprising SEQ ID NOS: 47, 48, 49, 50, 51, 52, 53, 54, 55, 56, 57, 58 or complements thereof. In specific embodiments, the labeled nucleic acid probe comprises a nucleic acid sequence comprising SEQ ID NOS: 23, 24, 25, 26, 27, 28, 29, 30, 31, 32, 33, 34, 35, 36, 37, 38, 39, 40, 41, 42, 43, 44, 45, 46 or variants or fragments thereof.

An active variant of any one of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57 or 58 can comprise a polynucleotide having at least 75%, 80% 85%, 90%, 91%, 92%, 93%, 94%, 95%, 96%, 97%, 98% or 99% sequence identity to SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57 or 58 as long as it is capable of amplifying and/or detecting the marker locus of interest. By “fragment” is intended a portion of the polynucleotide. A fragment or portion can comprise at least 10, 15, 20, 30, 40, 50, 75, 100, 150, 200, 250, 300, 350, 400 contiguous nucleotides of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57 or 58 as long as it is capable of amplifying and/or detecting the marker locus of interest.

Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; or any equivalent program thereof. By “equivalent program” is intended any sequence comparison program that, for any two sequences in question, generates an alignment having identical nucleotide residue matches and an identical percent sequence identity when compared to the corresponding alignment generated by GAP Version 10.

Traits or markers are considered to be linked if they co-segregate. A 1/100 probability of recombination per generation is defined as a map distance of 1.0 centiMorgan (1.0 cM). Genetic elements or genes located on a single chromosome segment are physically linked. Two loci can be located in close proximity such that recombination between homologous chromosome pairs does not occur between the two loci during meiosis with high frequency, e.g., such that linked loci co-segregate at least about 90% of the time, e.g., 91%, 92%, 93%, 94%, 95%, 96%, 97%, 98%, 99%, 99.5%, 99.75%, or more of the time. Genetic elements located within a chromosome segment are also genetically linked, typically within a genetic recombination distance of less than or equal to 50 centimorgans (cM), e.g., about 49, 40, 30, 20, 10, 5, 4, 3, 2, 1, 0.75, 0.5, or 0.25 cM or less. That is, two genetic elements within a single chromosome segment undergo recombination during meiosis with each other at a frequency of less than or equal to about 50%, e.g., about 49%, 40%, 30%, 20%, 10%, 5%, 4%, 3%, 2%, 1%, 0.75%, 0.5%, or 0.25% or less. Closely linked markers display a cross over frequency with a given marker of about 10% or less (the given marker is within about 10 cM of a closely linked marker). Put another way, closely linked loci co-segregate at least about 90% of the time. Genetic linkage as evaluated by recombination frequency is impacted by the chromatin structure of the region comprising the loci. Typically, the region is assumed to have a euchromatin structure during initial evaluations. However, some regions, such are regions closer to centrosomes, have a heterochromatin structure. Without further information, the predicted physical distance between genetic map positions is based on the assumption that the region is euchromatic, however if the region comprises heterochromatin the markers may be physically closer together. With regard to physical position on a chromosome, closely linked markers can be separated, for example, by about 1 megabase (Mb; 1 million nucleotides), about 500 kilobases (Kb; 1000 nucleotides), about 400 Kb, about 300 Kb, about 200 Kb, about 100 Kb, about 50 Kb, about 25 Kb, about 10 Kb, about 5 Kb, about 2 Kb, about 1 Kb, about 500 nucleotides, about 250 nucleotides, or less.

When referring to the relationship between two genetic elements, such as a genetic element contributing to tolerance and a proximal marker, “coupling” phase linkage indicates the state where the “favorable” allele at the tolerance locus is physically associated on the same chromosome strand as the “favorable” allele of the respective linked marker locus. In coupling phase, both favorable alleles are inherited together by progeny that inherit that chromosome strand. In “repulsion” phase linkage, the “favorable” allele at the locus of interest (e.g., a QTL for tolerance) is physically linked with an “unfavorable” allele at the proximal marker locus, and the two “favorable” alleles are not inherited together (i.e., the two loci are “out of phase” with each other).

Markers are used to define a specific locus on the soybean genome. Each marker is therefore an indicator of a specific segment of DNA, having a unique nucleotide sequence. Map positions provide a measure of the relative positions of particular markers with respect to one another. When a trait is stated to be linked to a given marker it will be understood that the actual DNA segment whose sequence affects the trait generally co-segregates with the marker. More precise and definite localization of a trait can be obtained if markers are identified on both sides of the trait. By measuring the appearance of the marker(s) in progeny of crosses, the existence of the trait can be detected by relatively simple molecular tests without actually evaluating the appearance of the trait itself, which can be difficult and time-consuming because the actual evaluation of the trait requires growing plants to a stage and/or under environmental conditions where the trait can be expressed. Molecular markers have been widely used to determine genetic composition in soybeans.

Favorable genotypes associated with at least trait of interest may be identified by one or more methodologies. In some examples one or more markers are used, including but not limited to AFLPs, RFLPs, ASH, SSRs, SNPs, indels, padlock probes, molecular inversion probes, microarrays, sequencing, and the like. In some methods, a target nucleic acid is amplified prior to hybridization with a probe. In other cases, the target nucleic acid is not amplified prior to hybridization, such as methods using molecular inversion probes (see, for example Hardenbol et al. (2003) Nat Biotech 21:673-678). In some examples, the genotype related to a specific trait is monitored, while in other examples, a genome-wide evaluation including but not limited to one or more of marker panels, library screens, association studies, microarrays, gene chips, expression studies, or sequencing such as whole-genome resequencing and genotyping-by-sequencing (GBS) may be used. In some examples, no target-specific probe is needed, for example by using sequencing technologies, including but not limited to next-generation sequencing methods (see, for example, Metzker (2010) Nat Rev Genet. 11:31-46; and, Egan et al. (2012) Am J Bot 99:175-185) such as sequencing by synthesis (e.g., Roche 454 pyrosequencing, IIlumina Genome Analyzer, and Ion Torrent PGM or Proton systems), sequencing by ligation (e.g., SOLiD from Applied Biosystems, and Polnator system from Azco Biotech), and single molecule sequencing (SMS or third-generation sequencing) which eliminate template amplification (e.g., Helicos system, and PacBio RS system from Pacific BioSciences). Further technologies include optical sequencing systems (e.g., Starlight from Life Technologies), and nanopore sequencing (e.g., GridION from Oxford Nanopore Technologies). Each of these may be coupled with one or more enrichment strategies for organellar or nuclear genomes in order to reduce the complexity of the genome under investigation via PCR, hybridization, restriction enzyme (see, e.g., Elshire et al. (2011) PLoS ONE 6:e19379), and expression methods. In some examples, no reference genome sequence is needed in order to complete the analysis.

The use of marker assisted selection (MAS) to select a soybean plant or germplasm which has a certain marker locus or marker profile is provided. For instance, in certain examples a soybean plant or germplasm possessing a certain predetermined favorable marker locus or haplotype will be selected via MAS. In certain other examples, a soybean plant or germplasm possessing a certain predetermined favorable marker profile will be selected via MAS.

Using MAS, soybean plants or germplasm can be selected for markers or marker alleles that positively correlate with tolerance to chloride salt stress, without actually raising soybean and measuring for tolerance (or, contrawise, soybean plants can be selected against if they possess markers that negatively correlate with tolerance). MAS is a powerful tool to select for desired phenotypes and for introgressing desired traits into cultivars of soybean (e.g., introgressing desired traits into elite lines). MAS is easily adapted to high throughput molecular analysis methods that can quickly screen large numbers of plant or germplasm genetic material for the markers of interest and is much more cost effective than raising and observing plants for visible traits. In some embodiments, the molecular markers or marker loci are detected using a suitable amplification-based detection method. In these types of methods, nucleic acid primers are typically hybridized to the conserved regions flanking the polymorphic marker region. In certain methods, nucleic acid probes that bind to the amplified region are also employed. In general, synthetic methods for making oligonucleotides, including primers and probes, are well known in the art. For example, oligonucleotides can be synthesized chemically according to the solid phase phosphoramidite triester method described by Beaucage and Caruthers (1981) Tetrahedron Letts 22:1859-1862, e.g., using a commercially available automated synthesizer, e.g., as described in Needham-VanDevanter, et al. (1984) Nucleic Acids Res. 12:6159-6168. Oligonucleotides, including modified oligonucleotides, can also be ordered from a variety of commercial sources known to persons of skill in the art.

It will be appreciated that suitable primers and probes to be used can be designed using any suitable method. It is not intended that the invention be limited to any particular primer, primer pair or probe. For example, primers can be designed using any suitable software program, such as LASERGENE® or Primer3.

It is not intended that the primers be limited to generating an amplicon of any particular size. For example, the primers used to amplify the marker loci and alleles herein are not limited to amplifying the entire region of the relevant locus. In some embodiments, marker amplification produces an amplicon at least 20 nucleotides in length, or alternatively, at least 50 nucleotides in length, or alternatively, at least 100 nucleotides in length, or alternatively, at least 200 nucleotides in length.

Non-limiting examples of polynucleotide primers useful for detecting the marker loci provided herein include those primers listed in Table 1, for example, SEQ ID NOS: 1, 2, 3, 4, 5, 6, 7, 8, 9, 10, 11, 12, 13, 14, 15, 16, 17, 18, 19, 20, 21 or 22.

PCR, RT-PCR, and LCR are in particularly broad use as amplification and amplification-detection methods for amplifying nucleic acids of interest (e.g., those comprising marker loci), facilitating detection of the markers. Details regarding the use of these and other amplification methods are well known in the art and can be found in any of a variety of standard texts. Details for these techniques can also be found in numerous references, such as Mullis, et al. (1987) U.S. Pat. No. 4,683,202; Arnheim & Levinson (1990) C&EN 36-47; Kwoh, et al. (1989) Proc. Natl. Acad. Sci. USA 86:1173; Guatelli, et al., (1990) Proc. Natl. Acad. Sci. USA87:1874; Lomell, et al., (1989) J. Clin. Chem. 35:1826; Landegren, et al., (1988) Science 241:1077-1080; Van Brunt, (1990) Biotechnology 8:291-294; Wu and Wallace, (1989) Gene 4:560; Barringer, et al., (1990) Gene 89:117, and Sooknanan and Malek, (1995) Biotechnology 13:563-564.

Such nucleic acid amplification techniques can be applied to amplify and/or detect nucleic acids of interest, such as nucleic acids comprising marker loci. Amplification primers for amplifying useful marker loci and suitable probes to detect useful marker loci or to genotype SNP alleles are provided. For example, exemplary primers and probes are provided in SEQ ID NOS: 1-46 and in Tables 1 and 2, and the design sequences are provided in SEQ ID NOS 47-58 and in Table 3. However, one of skill will immediately recognize that other primer and probe sequences could also be used. For instance primers to either side of the given primers can be used in place of the given primers, so long as the primers can amplify a region that includes the allele to be detected, as can primers and probes directed to other SNP marker loci. Further, it will be appreciated that the precise probe to be used for detection can vary, e.g., any probe that can identify the region of a marker amplicon to be detected can be substituted for those examples provided herein. Further, the configuration of the amplification primers and detection probes can, of course, vary. Thus, the compositions and methods are not limited to the primers and probes specifically recited herein.

In certain examples, probes will possess a detectable label. Any suitable label can be used with a probe. Detectable labels suitable for use with nucleic acid probes include, for example, any composition detectable by spectroscopic, radioisotopic, photochemical, biochemical, immunochemical, electrical, optical, or chemical means. Useful labels include biotin for staining with labeled streptavidin conjugate, magnetic beads, fluorescent dyes, radiolabels, enzymes, and colorimetric labels. Other labels include ligands, which bind to antibodies labeled with fluorophores, chemiluminescent agents, and enzymes. A probe can also constitute radiolabelled PCR primers that are used to generate a radiolabelled amplicon. Strategies for labeling nucleic acids and corresponding detection strategies can be found, e.g., in Haugland (1996) Handbook of Fluorescent Probes and Research Chemicals Sixth Edition by Molecular Probes, Inc. (Eugene Oreg.); or Haugland (2001) Handbook of Fluorescent Probes and Research Chemicals Eighth Edition by Molecular Probes, Inc. (Eugene Oreg.).

Detectable labels may also include reporter-quencher pairs, such as are employed in Molecular Beacon and TaqMan™ probes. The reporter may be a fluorescent organic dye modified with a suitable linking group for attachment to the oligonucleotide, such as to the terminal 3′ carbon or terminal 5′ carbon. The quencher may also be an organic dye, which may or may not be fluorescent, depending on the embodiment. Generally, whether the quencher is fluorescent or simply releases the transferred energy from the reporter by non-radiative decay, the absorption band of the quencher should at least substantially overlap the fluorescent emission band of the reporter to optimize the quenching. Non-fluorescent quenchers or dark quenchers typically function by absorbing energy from excited reporters, but do not release the energy radiatively.

Selection of appropriate reporter-quencher pairs for particular probes may be undertaken in accordance with known techniques. Fluorescent and dark quenchers and their relevant optical properties from which exemplary reporter-quencher pairs may be selected are listed and described, for example, in Berlman, Handbook of Fluorescence Spectra of Aromatic Molecules, 2nd ed., Academic Press, New York, 1971, the content of which is incorporated herein by reference. Examples of modifying reporters and quenchers for covalent attachment via common reactive groups that can be added to an oligonucleotide in the present invention may be found, for example, in Haugland, Handbook of Fluorescent Probes and Research Chemicals, Molecular Probes of Eugene, Oreg., 1992, the content of which is incorporated herein by reference.

In certain examples, reporter-quencher pairs are selected from xanthene dyes including fluoresceins and rhodamine dyes. Many suitable forms of these compounds are available commercially with substituents on the phenyl groups, which can be used as the site for bonding or as the bonding functionality for attachment to an oligonucleotide. Another useful group of fluorescent compounds for use as reporters are the naphthylamines, having an amino group in the alpha or beta position. Included among such naphthylamino compounds are 1-dimethylaminonaphthyl-5 sulfonate, 1-anilino-8-naphthalene sulfonate and 2-p-touidinyl-6-naphthalene sulfonate. Other dyes include 3-phenyl-7-isocyanatocoumarin; acridines such as 9-isothiocyanatoacridine; N-(p-(2-benzoxazolyl)phenyl)maleimide; benzoxadiazoles; stilbenes; pyrenes and the like. In certain other examples, the reporters and quenchers are selected from fluorescein and rhodamine dyes. These dyes and appropriate linking methodologies for attachment to oligonucleotides are well known in the art.

Suitable examples of reporters may be selected from dyes such as SYBR green, 5-carboxyfluorescein (5-FAM™ available from Applied Biosystems of Foster City, Calif.), 6-carboxyfluorescein (6-FAM), tetrachloro-6-carboxyfluorescein (TET), 2,7-dimethoxy-4,5-dichloro-6-carboxyfluorescein, hexachloro-6-carboxyfluorescein (HEX), 6-carboxy-2′,4,7,7′-tetrachlorofluorescein (6-TET™ available from Applied Biosystems), carboxy-X-rhodamine (ROX), 6-carboxy-4′,5′-dichloro-2′,7′-dimethoxyfluorescein (6-JOE™ available from Applied Biosystems), VIC™ dye products available from Molecular Probes, Inc., NED™ dye products available from Applied Biosystems, and the like. Suitable examples of quenchers may be selected from 6-carboxy-tetramethylrhodamine, 4-(4-dimethylaminophenylazo) benzoic acid (DABYL), tetramethylrhodamine (TAMRA), BHQ-0™, BHQ-1™, BHQ-2™, and BHQ-3™, each of which are available from Biosearch Technologies, Inc. of Novato, Calif., QSY-7™, QSY-9™, QSY-21™ and QSY-35™, each of which are available from Molecular Probes, Inc., and the like.

In one aspect, real time PCR or LCR is performed on the amplification mixtures described herein, e.g., using molecular beacons or TaqMan™ probes. A molecular beacon (MB) is an oligonucleotide which, under appropriate hybridization conditions, self-hybridizes to form a stem and loop structure. The MB has a label and a quencher at the termini of the oligonucleotide; thus, under conditions that permit intra-molecular hybridization, the label is typically quenched (or at least altered in its fluorescence) by the quencher. Under conditions where the MB does not display intra-molecular hybridization (e.g., when bound to a target nucleic acid, such as to a region of an amplicon during amplification), the MB label is unquenched. Details regarding standard methods of making and using MBs are well established in the literature and MBs are available from a number of commercial reagent sources. See also, e.g., Leone, et al. (1995) Nucl Acids Res. 26:2150-2155; Tyagi and Kramer (1996) Nat Biotechnol 14:303-308; Blok and Kramer (1997) Mol Cell Probes 11:187-194; Hsuih. et al. (1997) J Clin Microbiol 34:501-507; Kostrikis et al. (1998) Science 279:1228-1229; Sokol, et al. (1998) Proc. Natl. Acad. Sci. USA 95:11538-11543; Tyagi, et al. (1998) Nat Biotechnol 16:49-53; Bonnet, et al. (1999) Proc. Natl. Acad. Sci. USA 96:6171-6176; Fang, et al. (1999) J. Am. Chem. Soc. 121:2921-2922; Marras, et al. (1999) Genet. Anal. Biomol. Eng. 14:151-156; and Vet, et al. (1999) Proc. Natl. Acad. Sci. USA 96:6394-6399. Additional details regarding MB construction and use is found in the patent literature, e.g., U.S. Pat. Nos. 5,925,517; 6,150,097; and 6,037,130.

Another real-time detection method is the 5′-exonuclease detection method, also called the TaqMan™ assay, as set forth in U.S. Pat. Nos. 5,804,375; 5,538,848; 5,487,972; and 5,210,015, each of which is hereby incorporated by reference in its entirety. In the TaqMan™ assay, a modified probe, typically 10-25 nucleic acids in length, is employed during PCR which binds intermediate to or between the two members of the amplification primer pair. The modified probe possesses a reporter and a quencher and is designed to generate a detectable signal to indicate that it has hybridized with the target nucleic acid sequence during PCR. As long as both the reporter and the quencher are on the probe, the quencher stops the reporter from emitting a detectable signal. However, as the polymerase extends the primer during amplification, the intrinsic 5′ to 3′ nuclease activity of the polymerase degrades the probe, separating the reporter from the quencher, and enabling the detectable signal to be emitted. Generally, the amount of detectable signal generated during the amplification cycle is proportional to the amount of product generated in each cycle.

It is well known that the efficiency of quenching is a strong function of the proximity of the reporter and the quencher, i.e., as the two molecules get closer, the quenching efficiency increases. As quenching is strongly dependent on the physical proximity of the reporter and quencher, the reporter and the quencher are preferably attached to the probe within a few nucleotides of one another, usually within 30 nucleotides of one another, more preferably with a separation of from about 6 to 16 nucleotides. Typically, this separation is achieved by attaching one member of a reporter-quencher pair to the 5′ end of the probe and the other member to a nucleotide about 6 to 16 nucleotides away, in some cases at the 3′ end of the probe.

Separate detection probes can also be omitted in amplification/detection methods, e.g., by performing a real time amplification reaction that detects product formation by modification of the relevant amplification primer upon incorporation into a product, incorporation of labeled nucleotides into an amplicon, or by monitoring changes in molecular rotation properties of amplicons as compared to unamplified precursors (e.g., by fluorescence polarization).

Further, it will be appreciated that amplification is not a requirement for marker detection—for example, one can directly detect unamplified genomic DNA simply by performing a Southern blot on a sample of genomic DNA. Procedures for performing Southern blotting, amplification e.g., (PCR, LCR, or the like), and many other nucleic acid detection methods are well established and are taught, e.g., in Sambrook, et al., Molecular Cloning—A Laboratory Manual (3d ed.), Vol. 1-3, Cold Spring Harbor Laboratory, Cold Spring Harbor, N.Y., 2000 (“Sambrook”); Current Protocols in Molecular Biology, F. M. Ausubel, et al., eds., Current Protocols, a joint venture between Greene Publishing Associates, Inc. and John Wiley & Sons, Inc., (supplemented through 2002) (“Ausubel”)) and PCR Protocols A Guide to Methods and Applications (Innis, et al., eds) Academic Press Inc. San Diego, Calif. (1990) (Innis). Additional details regarding detection of nucleic acids in plants can also be found, e.g., in Plant Molecular Biology (1993) Croy (ed.) BIOS Scientific Publishers, Inc.

Other techniques for detecting SNPs can also be employed, such as allele specific hybridization (ASH). ASH technology is based on the stable annealing of a short, single-stranded, oligonucleotide probe to a completely complementary single-stranded target nucleic acid. Detection is via an isotopic or non-isotopic label attached to the probe. For each polymorphism, two or more different ASH probes are designed to have identical DNA sequences except at the polymorphic nucleotides. Each probe will have exact homology with one allele sequence so that the range of probes can distinguish all the known alternative allele sequences. Each probe is hybridized to the target DNA. With appropriate probe design and hybridization conditions, a single-base mismatch between the probe and target DNA will prevent hybridization.

Real-time amplification assays, including MB or TaqMan™ based assays, are especially useful for detecting SNP alleles. In such cases, probes are typically designed to bind to the amplicon region that includes the SNP locus, with one allele-specific probe being designed for each possible SNP allele. For instance, if there are two known SNP alleles for a particular SNP locus, “A” or “C,” then one probe is designed with an “A” at the SNP position, while a separate probe is designed with a “C” at the SNP position. While the probes are typically identical to one another other than at the SNP position, they need not be. For instance, the two allele-specific probes could be shifted upstream or downstream relative to one another by one or more bases. However, if the probes are not otherwise identical, they should be designed such that they bind with approximately equal efficiencies, which can be accomplished by designing under a strict set of parameters that restrict the chemical properties of the probes. Further, a different detectable label, for instance a different reporter-quencher pair, is typically employed on each different allele-specific probe to permit differential detection of each probe. In certain examples, each allele-specific probe for a certain SNP locus is 11-20 nucleotides in length, dual-labeled with a florescence quencher at the 3′ end and either the 6-FAM (6-carboxyfluorescein) or VIC (4,7,2′-trichloro-7′-phenyl-6-carboxyfluorescein) fluorophore at the 5′ end.

To effectuate SNP allele detection, a real-time PCR reaction can be performed using primers that amplify the region including the SNP locus, for instance the sequences listed in Table 3, the reaction being performed in the presence of all allele-specific probes for the given SNP locus. By then detecting signal for each detectable label employed and determining which detectable label(s) demonstrated an increased signal, a determination can be made of which allele-specific probe(s) bound to the amplicon and, thus, which SNP allele(s) the amplicon possessed. For instance, when 6-FAM- and VIC-labeled probes are employed, the distinct emission wavelengths of 6-FAM (518 nm) and VIC (554 nm) can be captured. A sample that is homozygous for one allele will have fluorescence from only the respective 6-FAM or VIC fluorophore, while a sample that is heterozygous at the analyzed locus will have both 6-FAM and VIC fluorescence.

The KASPar® and Illumina® Detection Systems are additional examples of commercially-available marker detection systems. KASPar® is a homogeneous fluorescent genotyping system which utilizes allele specific hybridization and a unique form of allele specific PCR (primer extension) in order to identify genetic markers (e.g. a particular SNP locus associated with chloride salt stress tolerance). Illumina® detection systems utilize similar technology in a fixed platform format. The fixed platform utilizes a physical plate that can be created with up to 384 markers. The Illumina® system is created with a single set of markers that cannot be changed and utilizes dyes to indicate marker detection.

These systems and methods represent a wide variety of available detection methods which can be utilized to detect markers associated with improved chloride salt stress tolerance, but any other suitable method could also be used.

Introgression of tolerance to chloride salt stress into non-tolerant or less-tolerant soybean germplasm is provided. Any method for introgressing a QTL or marker into soybean plants known to one of skill in the art can be used. Typically, a first soybean germplasm that contains tolerance to chloride salt stress derived from a particular marker locus or marker profile and a second soybean germplasm that lacks such tolerance derived from the marker locus or marker profile are provided. The first soybean germplasm may be crossed with the second soybean germplasm to provide progeny soybean germplasm. These progeny germplasm are screened to determine the presence of chloride salt stress tolerance derived from the marker locus or marker profile, and progeny that tests positive for the presence of tolerance derived from the marker locus or marker profile are selected as being soybean germplasm into which the marker locus or marker profile has been introgressed. Methods for performing such screening are well known in the art and any suitable method can be used.

One application of MAS is to use the tolerance markers, haplotypes or marker profiles to increase the efficiency of an introgression or backcrossing effort aimed at introducing a tolerance trait into a desired (typically high yielding) background. In marker assisted backcrossing of specific markers from a donor source, e.g., to an elite genetic background, one selects among backcross progeny for the donor trait and then uses repeated backcrossing to the elite line to reconstitute as much of the elite background's genome as possible.

Thus, the markers and methods can be utilized to guide marker assisted selection or breeding of soybean varieties with the desired complement (set) of allelic forms of chromosome segments associated with superior agronomic performance (resistance, along with any other available markers for yield, disease resistance, etc.). Any of the disclosed marker loci, marker alleles, haplotypes, or marker profiles can be introduced into a soybean line via introgression, by traditional breeding (or introduced via transformation, or both) to yield a soybean plant with superior agronomic performance. The number of alleles associated with tolerance that can be introduced or be present in a soybean plant ranges from 1 to the number of alleles disclosed herein, each integer of which is incorporated herein as if explicitly recited.

This also provides a method of making a progeny soybean plant and these progeny soybean plants, per se. The method comprises crossing a first parent soybean plant with a second soybean plant and growing the female soybean plant under plant growth conditions to yield soybean plant progeny. Methods of crossing and growing soybean plants are well within the ability of those of ordinary skill in the art. Such soybean plant progeny can be assayed for alleles associated with tolerance and, thereby, the desired progeny selected. Such progeny plants or seed can be sold commercially for soybean production, used for food, processed to obtain a desired constituent of the soybean, or further utilized in subsequent rounds of breeding. At least one of the first or second soybean plants is a soybean plant in that it comprises at least one of the marker loci or marker profiles, such that the progeny are capable of inheriting the marker locus or marker profile.

Often, a method is applied to at least one related soybean plant such as from progenitor or descendant lines in the subject soybean plants pedigree such that inheritance of the desired tolerance can be traced. The number of generations separating the soybean plants being subject to the methods provided herein will generally be from 1 to 20, commonly 1 to 5, and typically 1, 2, or 3 generations of separation, and quite often a direct descendant or parent of the soybean plant will be subject to the method (i.e., 1 generation of separation).

Genetic diversity is important for long term genetic gain in any breeding program. With limited diversity, genetic gain will eventually plateau when all of the favorable alleles have been fixed within the elite population. One objective is to incorporate diversity into an elite pool without losing the genetic gain that has already been made and with the minimum possible investment. MAS provides an indication of which genomic regions and which favorable alleles from the original ancestors have been selected for and conserved over time, facilitating efforts to incorporate favorable variation from exotic germplasm sources (parents that are unrelated to the elite gene pool) in the hopes of finding favorable alleles that do not currently exist in the elite gene pool.

For example, the markers, haplotypes, primers, probes, and marker profiles can be used for MAS in crosses involving elite×exotic soybean lines by subjecting the segregating progeny to MAS to maintain alleles, along with the tolerance marker alleles herein.

As an alternative to standard breeding methods of introducing traits of interest into soybean (e.g., introgression), transgenic approaches can also be used to create transgenic plants with the desired traits. In these methods, exogenous nucleic acids that encode a desired marker loci, marker profile or haplotype are introduced into target plants or germplasm. For example, a nucleic acid that codes for a tolerance trait is cloned, e.g., via positional cloning, and introduced into a target plant or germplasm.

Experienced plant breeders can recognize tolerant soybean plants in the field, and can select the tolerant individuals or populations for breeding purposes or for propagation. In this context, the plant breeder recognizes tolerant and non-tolerant or susceptible soybean plants. However, plant tolerance is a phenotypic spectrum consisting of extremes in tolerance and susceptibility, as well as a continuum of intermediate tolerance phenotypes. Evaluation of these intermediate phenotypes using reproducible assays are of value to scientists who seek to identify genetic loci that impart tolerance, to conduct marker assisted selection for tolerant populations, and to use introgression techniques to breed a tolerance trait into an elite soybean line, for example.

By improved tolerance is intended that the plants show a decrease in the disease symptoms that are the outcome of plant exposure to high concentrations of chloride salt. That is, the damage caused by the chloride salt stress is prevented, or alternatively, the disease symptoms caused by the chloride salt stress is minimized or lessened. Thus, improved tolerance to chloride salt stress can result in reduction of the disease symptoms by at least about 2% to at least about 6%, at least about 5% to about 50%, at least about 10% to about 60%, at least about 30% to about 70%, at least about 40% to about 80%, or at least about 50% to about 90% or greater. Hence, the methods provided herein can be utilized to protect plants from chloride salt stress. A tolerant plant, tolerant plant variety, or a plant or plant variety with improved tolerance will have a level of tolerance to chloride salt stress which is higher than that of a comparable susceptible plant or variety.

Screening and selection of tolerant soybean plants may be performed, for example, by exposing plants to chloride salt and selecting those plants showing tolerance to chloride salt stress. Various assays can be used to measure tolerance or improved tolerance to chloride salt stress. For example, the percentage of chloride in the plant can be measured by methods known in the art, or the plant can be examined for signs of chloride salt stress by visual inspection for symptoms such as leaf chlorosis, leaf scorching and stunting of plant growth. The severity of chloride salt stress can be scored, for example, by applying a scale. For example, a scale ranging from 1-9 can be used, with 1 representing a susceptible plant and 9 representing a tolerant plant. Such assays for screening soybean plants for chloride salt stress are well known in the art (see, e.g., Lee et al. (2008) Crop Sci 48:2194-2200). In addition, Examples 1 and 2 provided herein describe such assays for screening chloride salt stress phenotype.

The percentage of chloride in the plant can be measured by assays known in the art, including but not limited to a colorimetry, chromatography, potentiometry, and spectroscopy. One example of a colorimetric assay uses a mercuric thiocyanate reagent to detect chloride in a plant sample extract. An exemplary potentiometric assay precipitates chloride using a silver nitrate electrode and reagents. Chromatographic assays include ion chromatography of extracts, typically using FPLC or HPLC ion exchange columns. Spectroscopic methods include inductively coupled plasma optical emission spectroscopy (ICP-OES or ICP). An example of how ICP is used to quantify chloride from soybean tissue is provided in Example 4.

In some examples, a kit or an automated system for detecting marker loci, haplotypes, and marker profiles, and/or correlating the marker loci, haplotypes, and marker profiles with a desired phenotype (e.g., tolerance to chloride salt stress) are provided. As used herein, “kit” refers to a set of reagents for the purpose of performing the various methods of detecting or identifying herein, more particularly, the identification and/or the detection of a soybean plant or germplasm having tolerance to chloride salt stress.

In one embodiment, a kit for detecting or selecting at least one soybean plant or soybean germplasm with tolerance to chloride salt stress is provided. Such a kit comprises (a) primers or probes for detecting one or more marker loci associated with chloride salt stress tolerance, wherein at least one of the primers and probes in the kit are capable of detecting a marker locus comprising GM03:40563114, GM03:40576895, GM03:40489573, GM03:40489574, GM03:40557669, GM03:40591130, GM03:40703866, GM03:40554209, GM03:40589164, GM03:40606905, GM03:40632077, GM03:40705541, GM03:40576921, S06578-1-A, S16256-001-Q001, S16255-001-Q001, S16254-001-Q001, S16253-001-Q001, S16252-001-Q001, S16232-001-Q001 S12869-1-Q1, S00145-1-A, S16226-001-K001, S16227-001-K001, S04733-1-A or a marker closely linked thereto; and (b) instructions for using the primers or probes for detecting the one or more marker loci and correlating the detected marker loci with predicted tolerance to chloride salt stress.

Thus, a typical kit or system can include a set of marker probes or primers configured to detect at least one favorable allele of one or more marker locus associated with tolerance to chloride salt stress, for instance a favorable marker locus, haplotype or marker profile. These probes or primers can be configured, for example, to detect the marker loci noted in the tables and examples herein, e.g., using any available allele detection format, such as solid or liquid phase array based detection, microfluidic-based sample detection, etc. The systems and kits can further include packaging materials for packaging the probes, primers, or instructions, controls such as control amplification reactions that include probes, primers or template nucleic acids for amplifications, molecular size markers, or the like.

A typical system can also include a detector that is configured to detect one or more signal outputs from the set of marker probes or primers, or amplicon thereof, thereby identifying the presence or absence of the allele. A wide variety of signal detection apparatus are available, including photo multiplier tubes, spectrophotometers, CCD arrays, scanning detectors, phototubes and photodiodes, microscope stations, galvo-scans, microfluidic nucleic acid amplification detection appliances and the like. The precise configuration of the detector will depend, in part, on the type of label used to detect the marker allele, as well as the instrumentation that is most conveniently obtained for the user. Detectors that detect fluorescence, phosphorescence, radioactivity, pH, charge, absorbance, luminescence, temperature, magnetism or the like can be used. Typical detector examples include light (e.g., fluorescence) detectors or radioactivity detectors. For example, detection of a light emission (e.g., a fluorescence emission) or other probe label is indicative of the presence or absence of a marker allele. Fluorescent detection is generally used for detection of amplified nucleic acids (however, upstream and/or downstream operations can also be performed on amplicons, which can involve other detection methods). In general, the detector detects one or more label (e.g., light) emission from a probe label, which is indicative of the presence or absence of a marker allele. The detector(s) optionally monitors one or a plurality of signals from an amplification reaction. For example, the detector can monitor optical signals which correspond to real time amplification assay results.

System or kit instructions that describe how to use the system or kit or that correlate the presence or absence of the favorable allele with the predicted tolerance are also provided. For example, the instructions can include at least one look-up table that includes a correlation between the presence or absence of the favorable alleles, haplotypes, or marker profiles and the predicted tolerance. The precise form of the instructions can vary depending on the components of the system, e.g., they can be present as system software in one or more integrated unit of the system (e.g., a microprocessor, computer or computer readable medium), or can be present in one or more units (e.g., computers or computer readable media) operably coupled to the detector. As noted, in one typical example, the system instructions include at least one look-up table that includes a correlation between the presence or absence of the favorable alleles and predicted tolerance. The instructions also typically include instructions providing a user interface with the system, e.g., to permit a user to view results of a sample analysis and to input parameters into the system.

Isolated polynucleotides comprising the nucleic acid sequences of the primers and probes provided herein are also encompassed herein. In specific embodiments, the isolated polynucleotide comprises SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46 or variants or fragments thereof. In other embodiments, the isolated polynucleotide comprises a polynucleotide having at least 90% sequence identity to SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45 or 46. In yet other embodiments, the isolated polynucleotide comprises a polynucleotide comprising at least 10 contiguous nucleotides of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45 or 46.

In certain embodiments, the isolated nucleic acids are capable of hybridizing under stringent conditions to nucleic acids of a soybean cultivar tolerant to chloride salt stress, for instance to particular SNPs that comprise a marker locus, haplotype or marker profile.

As used herein, a substantially identical or complementary sequence is a polynucleotide that will specifically hybridize to the complement of the nucleic acid molecule to which it is being compared under high stringency conditions. A polynucleotide is said to be the “complement” of another polynucleotide if they exhibit complementarity. As used herein, molecules are said to exhibit “complete complementarity” when every nucleotide of one of the polynucleotide molecules is complementary to a nucleotide of the other. Two molecules are said to be “minimally complementary” if they can hybridize to one another with sufficient stability to permit them to remain annealed to one another under at least conventional “low-stringency” conditions. Similarly, the molecules are said to be “complementary” if they can hybridize to one another with sufficient stability to permit them to remain annealed to one another under conventional “high-stringency” conditions.

TABLE 1
Primer Sequences.

| | SNP | | Forward Primer | Reverse Primer | Allele | Allele |
|---|---|---|---|---|---|---|
| LG (ch) | position | Marker | Sequence | Sequence | 1 | 2 |
| N (Gm03) | 40632077 | S16256-001- | ATGCTTTACATTGCTAT | CGTAGTTGACAAGTTAG | C | G |
| | | Q001 | TGATGTAGTAGT (SEQ | TTAAAAGGTAAAA (SEQ | | |
| | | | ID NO: 1) | ID NO: 2) | | |
| N (Gm03) | 40557669 | S16255-001- | AAGCATGAATTATTGGA | GGCAAATGCTAATGTTG | T | C |
| | | Q001 | TTTTGTTAAT (SEQ ID | GTGT (SEQ ID NO: 4) | | |
| | | | NO: 3) | | | |
| N (Gm03) | 40703866 | S16254-001- | AAGATCACACATATGAG | TTTGGATTTTGGAGTAT | A | G |
| | | Q001 | CAAGTAGGC (SEQ ID | GAATGAA (SEQ ID NO: 6) | | |
| | | | NO: 5) | | | |
| N (Gm03) | 40563114 | S16253-001- | TGCGCATTAAATATACA | TCCAATTTTACCCTTTAT | A | G |
| | | Q001 | TTAGAAATTTTAG (SEQ | TCTTACGA (SEQ ID | | |
| | | | ID NO: 7) | NO: 8) | | |
| N (Gm03) | 40576895 | S16252-001- | CTCACTCGAGTAAAACA | CTACCATTATTA (SEQ ID | T | C |
| | | Q001 | CAGCAAGAAGCCGCAA | NO: 10) | | |
| | | | GT (SEQ ID NO: 9) | | | |
| N (Gm03) | 40606905 | S16232-001- | GACGCCAAATAGAAGC | TGTGTTAGACTGACGTG | T | C |
| | | Q001 | GATAGTAA (SEQ ID | ATAACCA (SEQ ID | | |
| | | | NO: 11) | NO: 12) | | |
| N (Gm03) | 37017850 | S04733-1-A | CAGTTGCCACAGGAGTT | GGGCTGGATAGGTTCTT | T | C |
| | | | GC (SEQ ID NO: 13) | CAA (SEQ ID NO: 14) | | |
| N (Gm03) | 39796244 | S00145-1-A | GTGCAAAAAGCAAACC | GCAAAGAGTGACACTTA | A | C |
| | | | CTGTGG (SEQ ID NO: 15) | AGCAGTGCAA (SEQ ID | | |
| | | | | NO: 16) | | |
| N (Gm03) | 40061269 | S16227-001- | | GGATATGGAAAGCAAC | G | A |
| | | K001 | | AAACTTTCTGGTA (SEQ | | |
| | | | | ID NO: 17) | | |
| N (Gm03) | 38806917 | S12869-1-Q1 | CCCGTCATGAACCATAC | TCTTTCATGTTTGGCAC | A | G |
| | | | ACA (SEQ ID NO: 18) | AGC | | |
| | | | | (SEQ ID NO: 19) | | |
| N (Gm03) | 40061119 | S16226-001- | | CCAAAGTCCTTGAGAAA | T | C |
| | | K001 | | TTGTTCATCACTA (SEQ | | |
| | | | | ID NO: 20) | | |
| N (Gm03) | 40672157 | S06578-1-Q2 | CAATTTTGACCAATATT | GTGGTGAACCTTGTCGT | C | G |
| | | | TCCAGTTC (SEQ ID NO: | GAA (SEQ ID NO: 22) | | |
| | | | 21) | | | |

TABLE 2
Probe Sequences.

| | SNP | | | | | |
|---|---|---|---|---|---|---|
| LG (ch) | position | Marker | Probe 1 | Probe 1 Sequence | Probe 2 | Probe 2 Sequence |
| N (Gm03) | 40632077 | S16256-001- | S16256-001- | TCATCTAGTTCA | S16256-001- | TCATCTAGTTGA |
| | | Q001 | X001 | ATGGT (SEQ ID | X002 | ATGGT (SEQ ID |
| | | | | NO: 23) | | NO: 24) |
| N (Gm03) | 40557669 | S16255-001- | S16255-001- | TTACACATACCA | S16255-001- | ATTTTACACACA |
| | | Q001 | X001 | ATAAA (SEQ ID | X002 | CCAATAA (SEQ ID |
| | | | | NO: 25) | | NO: 26) |
| N (Gm03) | 40703866 | S16254-001- | S16254-001- | CGGTTGGACTAT | S16254-001- | CGGTTGGACTAC |
| | | Q001 | X001 | TGA (SEQ ID | X002 | TGAT (SEQ ID |
| | | | | NO: 27) | | NO: 28) |
| N (Gm03) | 40563114 | S16253-001- | S16253-001- | CATAATTGACTC | S16253-001- | ATAACATAATTG |
| | | Q001 | X001 | ATTAAC (SEQ ID | X002 | GCTCATT (SEQ ID |
| | | | | NO: 29) | | NO: 30) |
| N (Gm03) | 40576895 | S16252-001- | S16252-001- | CATAAACCAAAA | S16252-001- | AACATAAACCAG |
| | | Q001 | X001 | ACTG (SEQ ID | X002 | AAACT (SEQ ID |
| | | | | NO: 31) | | NO: 32) |
| N (Gm03) | 40606905 | S16232-001- | S16232-001- | ACGACACCATAT | S16232-001- | ACGACACCGTAT |
| | | Q001 | X003 | TGA (SEQ ID | X004 | TGA (SEQ ID |
| | | | | NO: 33) | | NO: 34) |
| N (Gm03) | 37017850 | S04733-1-A | S04733-1-P1 | ACCATTAGAAAC | S04733-1-P2 | ACCATTAGGAAC |
| | | | | TCG (SEQ ID NO: | | TCG (SEQ ID NO: |
| | | | | 35) | | 36) |
| N (Gm03) | 39796244 | S00145-1-A | S00145-1-P1 | TGCAATTCTaTTA | S00145-1-P2 | CAATTCTcTTAAG |
| | | | | AGCC (SEQ ID NO: | | CCC (SEQ ID NO: |
| | | | | 37) | | 38) |
| N (Gm03) | 40061269 | S16227-001- | S16227-1-P1 | GAAGGTGACCAA | S16227-1-P2 | GAAGGTCGGAGT |
| | | K001 | | GTTCATGCTAATT | | CAACGGATTCCT |
| | | | | GACTCAACTTAC | | AATTGACTCAAC |
| | | | | TTACCTCAG (SED | | TTACTTACCTCAA |
| | | | | ID NO: 39) | | (SEQ ID NO: 40) |
| N (Gm03) | 38806917 | S12869-1- | S12869-1-P1 | TTTTAACCATGCT | S12869-1-P2 | TTTAGCCATGCTT |
| | | Q1 | | TTTGG (SED ID | | TTG (SEQ ID NO: |
| | | | | NO: 41) | | 42) |
| N (Gm03) | 40061119 | S16226-001- | S16226-001-P1 | GAAGGTGACCAA | S16226-001-P2 | GAAGGTCGGAGT |
| | | K001 | | GTTCATGCTATCT | | CAACGGATTCTG |
| | | | | GCGATGTTTCCA | | CGATGTTTCCAG |
| | | | | GTTAACTGATT | | TTAACTGATC |
| | | | | (SED ID NO: 43) | | (SEQ ID NO: 44) |
| N (Gm03) | 40672157 | S06578-1- | S06578-1-P1 | CCAAGCTAACAT | S06578-1-P2 | CCAAGCTAAGAT |
| | | Q2 | | GTC (SED ID NO: | | GTC (SEQ ID NO: |
| | | | | 45) | | 46) |

TABLE 3
Sequences Comprising Marker Loci.

| | SNP | Marker | Sequence | |
|---|---|---|---|---|
| LG (ch) | position | Name | Name | Design Sequence |
| N (Gm03) | 40632077 | S16256- | gmchr3v1: | AAATATTTACCAACACATGGTGCCGGTGAGAGAATCCTTGAC |
| | | 001-Q001 | 40632077_ | GCTTGGAGTGTTAATGAACACCAAAGGGTTGGTGGAGCTAA |
| | | | 201 | TCGTTCTCAATATTGGCAGAGAGAAGAAGGTGAGTTATGCTT |
| | | | | TACATTGCTATTGATGTAGTAGTAATTATACAAATATGTTGTT |
| | | | | TTGTGTTATTCTACGATTATCATCATCTAGTTCAATGGTTAAC |
| | | | | TTTATCACATATTTATAGTTGAATATAAGTTAGTTTATTAATT |
| | | | | TTCAACTAAAAATTAACTTTTTTTACCTTTTAACTAACTTGTC |
| | | | | AACTACGTATCTTTTTGTCGTACACACTATGAGGTGTCACTTA |
| | | | | TTTTCATAGACTCTTATGAGAATAGTACATACACACACGAAA |
| | | | | TTTAACTTTTTTCTTTAAA (SEQ ID NO: 47) |
| N (Gm03) | 40557669 | S16255- | gmchr3v1: | CTTAACTAATATCCTATAAACACTAGTTAACATTTGCCAATA |
| | | 001-Q001 | 40557669_ | ATTTTTACATGTTATTACTTTGGTAAAATCATTRTATAACTAA |
| | | | 201 | GCATGAATTATTGGATTTTGTTAATTAGTGTTTTTTGACATTG |
| | | | | ATTAAGAAATTAAAAGGAAAAAATATTTATTATGGAGAGGC |
| | | | | ATAAAAAAAACAAATAATATAATTTTACACATACCAATAAA |
| | | | | AATCTTTCTCTTTTTAATTCCTAAACCAATACACCAACATTAG |
| | | | | CATTTGCCAAGGTTGTTTGTCTCAAAAAATTATGGAAAGTAC |
| | | | | CTTTTGAAGGTAACATTCCCTTATCCACAAATAGCTTAGTCA |
| | | | | TGTCAAATTGTTGTACTGCCATCCATATACATCATGCCTTAAT |
| | | | | TAGTCCAAGACACCTCAATCA (SEQ ID NO: 48) |
| N (Gm03) | 40703866 | S16254- | gmchr3v1: | ACAGAGAAAATTGAATATAAACAAGCAGAACTGTAAAATAG |
| | | 001-Q001 | 40703866_ | GAATCACTTCAAAATAGAGTGCAATTAGGTTCAAACAAATGT |
| | | | 201 | GAAGTTACCAATGAAATGCAAAGTAAGATCACACATATGAG |
| | | | | CAAGTAGGCTCGGGGCCTCAAAAACCAAAGTTATGAAACCC |
| | | | | GGTCCRATCATCAACCCGCTTGARGTAGTGGATCAATAGTCC |
| | | | | AACCGGTGGGTCAATAATTCATCTGGTATATATTAAAAAATT |
| | | | | TAAAATTATATATGTATCTATTATATATAAGTATATAACTAA |
| | | | | CATTATTTGATTAAGAAAARTTCATTCATACTCCAAAATCCA |
| | | | | AAATAACAATTAAAGTATTAAAGTTAATAACACAAGTCCAC |
| | | | | AAATAATAATTCAATAACACAATTACA (SEQ ID NO: 49) |
| N (Gm03) | 40563114 | S16253- | gmchr3v1: | ACTCTTACCAAAATATATATTAAATTTTTTAATAGTAAATATT |
| | | 001-Q001 | 40563114_ | TTTTATTTTTAAAAAAATATCRGATTAAATAATTCGTATGATT |
| | | | 201 | TATATCAAAATTAATTGTCACAAAATTTATTATTTAAATTTAY |
| | | | | ATGCGCATTAAATATACATTAGAAATTTTAGTATTATGTATA |
| | | | | GCAACATTATTTATTTTATAACATAATTGACTCATTAACTCTT |
| | | | | ACGAAAGAAGTTYTTTCGTAAGAATAAAGGGTAAAATTGGA |
| | | | | AAAATGAGAAAATACTGGGTGCACCAACAATAATGCTGGGT |
| | | | | GCACCTAGCAACACCCAAACTTGTAAGAAAGACTTGGACAA |
| | | | | AAAAAAAATTGCAAACCAAGACCATTAGGGCAAAATATAAG |
| | | | | GCTAGCAAAAAAACACCTTGACC (SEQ ID NO: 50) |
| N (Gm03) | 40576895 | S16252- | gmchr3v1: | AGCCCGACGGATTAAACTRAGTTTATCCGAGTTGATTGCATA |
| | | 001-Q001 | 40576895_ | ATCATAATTAATGGAAACTGAGAAGTAAAAAATAAGTAAAT |
| | | | 201 | TTTAATGTGCATAATTTAWTTTTTTTGTAAAAAGAAAAGAAA |
| | | | | CAGAAAAAACTAACTACTACCTGGGAAACGCAACTCCTGCT |
| | | | | GCATCAGCAAGAAGCCGCAAGTTGAGATCAGTTTTTGGTTTA |
| | | | | TGTTACCCACCCAATAATGTGCATAACTGTATAATAGAATAC |
| | | | | AGTTTGCAAATTTTATTTAAAAAAACTAGAGTTTTTAATTTG |
| | | | | AGTTTAAATCTTCAATATGGACTTATGTTAAACATAAGGTAA |
| | | | | GAGAATTTTATTGTTTATAAGTAATAATGGTAGTGTTTTACTC |
| | | | | GAGTGAGATTACATCCTTAAGATA (SEQ ID NO: 51) |
| N (Gm03) | 40606905 | S16232- | gmchr3v1: | CACAAATCTAACTAATATAAAATATTTAAGTAAARAAAAGG |
| | | 001-Q001 | 40606905_ | TAAGGTTGCCTATAATTATAAAAAATTAAAAATTAGGTAGTG |
| | | | 201 | AACAAGGACTCGRCACATAAAAATTGAGAGCATTTATTTCTT |
| | | | | TTTCTTGACGCCAAATAGAAGCGATAGTAATGTTTGTAAGTA |
| | | | | TTATATARGCTTTATTGTGACAYAATGTCAATATGGTGTCGT |
| | | | | GGTGTAGTTGGTTATCACGTCAGTCTAACACACTGAAGGTCT |
| | | | | CCGGTTCGAGTCCGGGCGACGCCATTATATTCTATAACATTT |
| | | | | AATTTTACACTTCTACACATATTTTTGGGTCAGAAGCTTTACA |
| | | | | TTATAGGCTAGCCCACAAGATGGAAAGAGAAAGCATGCAGG |
| | | | | CCCAAATGAACAAAATGTAATTAC (SEQ ID NO: 52) |
| N Gm03) | 37017850 | S047331- | | AATGAACTTACTATCATAAAGGTTGAGCTAATTTTATATATA |
| | | A | | TCAnCnnTGCCTCTCCCCTAGAATTCTCAAATTGAGAGGGTTA |
| | | | | ACCAAAATGAATGTGCAGTTAACTAACTAACTACAAGGAAA |
| | | | | ATGCCAAACAGAAGAAAATAACTAATAAAAGAGGCATTTTC |
| | | | | ATTTTGAATTAGAAGTATAAAACAGTTCAGTACATCCAGAAC |
| | | | | AGAACGTTTTCCAATGGTATCTAAGTTGTTAAATGTGGTCAC |
| | | | | TATTGTTTTTCTTTTAAGGGCTGGATAGGTTCTTCAAATCCTC |
| | | | | TTCCTTTGCATACCAGCTTGGTATTATTTTTGAAACATGGGGA |
| | | | | TAAAGATCCTTGTACGAGTTYCTAATGGTTCCTTCTGCAACT |
| | | | | CCTGTGGCAACTGATATATCTGCACCAACACACTAGGTCAGA |
| | | | | TACAAATATGCAATAGCTCATACATGTGAATATTCTTTTAGA |
| | | | | AACCTATTTGCAATCAAATTTAACTGCAAATTCAAAATTCAC |
| | | | | CACACAAGAAATCAGAGAGACGATTTTCATGAAGAATTAAA |
| | | | | CCAATCAAATATGTTTGTATTTCATTGCTTTCATGTTTCACCA |
| | | | | TAAGAAGTGTGGCACATGGTCATAGCtgT (SED ID NO: 53) |
| N (Gm03) | 39796244 | S00145-1- | | AAGAAAAGGGAGGTTGTGGTTGAGAAGACTGGTGGCCCGGC |
| | | A | | TGAGAGCTATGACGATTTTGCTGCATCTTTGCCGGAGAATGA |
| | | | | TTGCAGATATGCTGTCTTTGACTATGATTTCGAAGAAAAGGG |
| | | | | AGGTTGTGGTTGAGAAGACTGGTGGCCCGGCTGAGAGCTAT |
| | | | | GACGATTTTGCTGCATCTTTGCCGGAGAATGATTGCAGATAT |
| | | | | GCTGTCTTTGACTATGATTTCGGCATTGTTGCACCAACCAGC |
| | | | | AAGATGTTTGTGAGGCATGATACATGATAGGCATTTTCTTGT |
| | | | | GCAAAAAGCAAACCCTGTGGCATATATGATCATATCATATGT |
| | | | | TTGGCAGTTTCAAAATAACCATGCCATGCAATTCTMTTAAGC |
| | | | | CCATGTCAATATTGCACTGCTTAAGTGTCACTCTTTGCTTGTT |
| | | | | CTCTGATTATGGAGCTACATGTTCATTGTTTGTTGTCTTGGAT |
| | | | | TAAATTTCTTGTCTTATTCTTTGGACACATTGTTTTTACATGA |
| | | | | TTGGGCTGTCAATCTCACCTAGTAGAATAA (SED ID NO: 54) |
| N (Gm03) | 40061269 | S16227- | gmchr3v1: | CAAAGTCCTTGAGAAATTGTTCATCACTATTTAGAGTCTGGC |
| | | 001-K001 | 40061269_ | TTGATGATRATCAGTTAACTGGAAACATCGCAGATGCATTTG |
| | | | 201 | GAGTACTCCCAGMTGCAGAAATAAGTTGGTTGGTGAGCYCT |
| | | | | CCTGGGAGTGGGGTGAATGTGTGAACTTAACTCAAATGGAT |
| | | | | ATGGAAAGCAACAAACTTTCTGGTAAAATTCCATTTGAGGTA |
| | | | | AGTAAGTTGAGTCAATTAGGGCATCTAAGCCWGCATTCCAA |
| | | | | TGAATTCTGTAAYATTCCATTTTTTATAAATTAATTTAAAAAG |
| | | | | AATTGTTATTTATAAATAAATAGAGTTTTAGAAAAATGATGA |
| | | | | GGTTTTTGTAATTAAATAAATAAGGAAAAATAACTTTATTAA |
| | | | | AATAATAATGATTTGAGAGAAAATA (SED ID NO: 55) |
| N (Gm03) | 38806917 | S12869-1- | | AAGTTGAGGGRGTTAGGGGTSGAAGTSATGATGGTGCAGTCT |
| | | Q1 | | TGGGTTAAGGATGATGGAGTGTTTGTGGCGGAGATGAGAGC |
| | | | | CATGGTGAGGGAAAATGGTAACGGGATAAAGGCTAGTGTTA |
| | | | | TWGAAGTGAAAAATGCCCTTAATCAGATCATACCCCGTCAT |
| | | | | GAACCATACACACTTGCTTCCAGTGATCATTTTTARCCATGC |
| | | | | TTTTGGACTAAGGTGAGACGGCTGTGCCAAACATGAAAGAT |
| | | | | GTGTGTTAAATGTTAATTGAATAAGTTTGTGTAAGAATTTAA |
| | | | | CTCATTCTATGTTTGGTCAAYTAAGTACGCGAATTGATCATG |
| | | | | TAAAATATCATGGACTTGCAGGGGGTGGAGGGTTGTGCCAT |
| | | | | ACCCATGATTGTCCTCCTTAATTAGCCT (SED ID NO: 56) |
| N (Gm03) | 40061119 | S16226- | gmchr3v1: | GCATTCCCAGAGAATTTGGAAAGAGTAATCCTTCTTTGACTC |
| | | 001-K001 | 40061119_ | ATGTCTACCTTTCAAACAGCTTCTCTGGAGAACTGCATCCTG |
| | | | 201 | ACTTGTGCAGTGATGGTAAGCTAGTTATTTTGGCAGTCAATA |
| | | | | ACAACAGCTTTTCAGGSCCATTGCCAAAGTCCTTGAGAAATT |
| | | | | GTTCATCACTATTTAGAGTCTGGCTTGATGATAATCAGTTAA |
| | | | | CTGGAAACATCGCAGATGCATTTGGAGTACTCCCAGMTGCA |
| | | | | GAAATAAGTTGGTTGGTGAGCYCTCCTGGGAGTGGGGTGAA |
| | | | | TGTGTGAACTTAACTCAAATGGATATGGAAAGCAACAAACTT |
| | | | | TCTGGTAAAATTCCATYTGAGGTAAGTAAGTTGAGTCAATTA |
| | | | | GGGCATCTAAGCCWGCATTCCAATG (SED ID NO: 57) |
| N (Gm03) | 40672157 | S06578-1- | | GCCAAAAAGGTAGCATATAACTTCATTGAAGTTCATGCCAAA |
| | | Q2 | | TAGACCTGAAAACGGGTCCCTTCAAAGTTCACATAAGTGAAC |
| | | | | ACTAGCCAGAAATAAGAGTGTAACCTTTATAAAGAAATTTTT |
| | | | | GTCCCCAGGAATTAGGTAAGGCATTCAAAGAAATCCAATTTG |
| | | | | ATAACAGCTATAATTTGGTTTTTGCTTAAAGTAGCATTGCCA |
| | | | | GGAACATAAATTTAGGGTACAACAAAGTGCAAGATATAATT |
| | | | | AAACCAAATTAAATCTGCAGGTTGGATATGAACTAATGGTCT |
| | | | | CAATCAATTCTCTCTTCTTCCCTTTATATGTATAAACAATAAA |
| | | | | CATTTTTTTTTTCAATTTTGACCAATATTTCCAGTTCTATGTTT |
| | | | | ACAATGTTTTGCCAAGCTAASATGTCTTCCACAATTTCTAAAT |
| | | | | TTTTCTGATCTACATTCACGACAAGGTTCACCACTGCTTTTTT |
| | | | | GAATAAAAAGTGGAGCCTCATATCACACATATCCAAAGTTTT |
| | | | | CTTAGACCCACATATCTGCATCATGTCATTATTAATCCTATTG |
| | | | | AGTACCACTGTCCAAATGGGATGACTTGACCTTAGAGGTGAT |
| | | | | CTTTGAGTTTCAAAGGGGTATTTGTACAAGAAACCTCATTCA |
| | | | | ACAATTTATGTTTCCAAACCACCTCCCCAAACCAGAGAAGCA |
| | | | | AGAGAAAACACTGAAATGCAAAATGTTCAGCAGAAAATGTA |
| | | | | TTTTTCTTCTCCTTTACTCCCTTCCCTCATGATTAAAAAAAGT |
| | | | | CTTTTTGAGGAATCATCTGTGGATGGATTGCAAATTGAC |
| | | | | (SED ID NO: 58) |

Non-limiting examples of methods and compositions disclosed herein are as follows:

1. A method of identifying a first soybean plant or a first soybean germplasm that displays tolerance to chloride salt stress, the method comprising detecting in the genome of said first soybean plant or in the genome of said first soybean germplasm at least one marker locus that is associated with the tolerance, wherein the at least one marker locus comprises GM03:40563114, GM03:40576895, GM03:40489573, GM03:40489574, GM03:40557669, GM03:40591130, GM03:40703866, GM03:40554209, GM03:40589164, GM03:40606905, GM03:40632077, GM03:40705541, GM03:40576921, S06578-1-A, S16256-001-Q001, S16255-001-Q001, S16254-001-Q001, S16253-001-Q001, S16252-001-Q001, S16232-001-Q001 or a marker closely linked thereto.
2. The method of embodiment 1, wherein at least two or more of the marker loci are detected.
3. The method of embodiment 1, wherein the germplasm is a soybean variety.
4. The method of embodiment 1, wherein the method further comprises selecting the first soybean plant or first soybean germplasm or a progeny thereof having the at least one marker locus.
5. The method of embodiment 4, further comprising crossing the selected first soybean plant or first soybean germplasm with a second soybean plant or second soybean germplasm.
6. The method of embodiment 5, wherein the second soybean plant or second soybean germplasm comprises an exotic soybean strain or an elite soybean strain.
7. The method of any one of embodiments 1-6, wherein the detecting comprises DNA sequencing of at least one of said marker loci.
8. The method of any one of embodiments 1-6, wherein the detecting comprises amplifying at least one of said marker loci and detecting the resulting amplified marker amplicon.
9. The method of embodiment 8, wherein the amplifying comprises:
a) admixing an amplification primer or amplification primer pair for each marker locus being amplified with a nucleic acid isolated from the first soybean plant or the first soybean germplasm, wherein the primer or primer pair is complementary or partially complementary to a variant or fragment of the genomic region comprising the marker locus, and is capable of initiating DNA polymerization by a DNA polymerase using the soybean nucleic acid as a template; and
b) extending the primer or primer pair in a DNA polymerization reaction comprising a DNA polymerase and a template nucleic acid to generate at least one amplicon.
   10. The method of embodiment 9, wherein said method comprises amplifying a variant or fragment of one or more polynucleotides comprising SEQ ID NOs: 47, 48, 49, 50, 51, 52 or 58.
   11. The method of embodiment 9, wherein said primer or primer pair comprises a variant or fragment of one or more polynucleotides comprising SEQ ID NOs: 47, 48, 49, 50, 51, 52, 58 or complements thereof.
   12. The method of embodiment 11, wherein said primer or primer pair comprises a nucleic acid sequence comprising SEQ ID NOs: 1, 2, 3, 4, 5, 6, 7, 8, 9, 10, 11, 12, 21, 22 or variants or fragments thereof.
   13. The method of embodiment 9, wherein the method further comprises providing one or more labeled nucleic acid probes suitable for detection of each marker locus being amplified.
   14. The method of embodiment 13, wherein said labeled nucleic acid probe comprises a nucleic acid sequence comprising a variant or fragment of one or more polynucleotides comprising SEQ ID NOs: 47, 48, 49, 50, 51, 52, 58 or complements thereof.
   15. The method of embodiment 14, wherein the labeled nucleic acid probe comprises a nucleic acid sequence comprising SEQ ID NOs: 23, 24, 25, 26, 27, 28, 29, 30, 31, 32, 33, 34, 45 or 46.
   16. An isolated polynucleotide capable of detecting a marker locus of the soybean genome comprising GM03:40563114, GM03:40576895, GM03:40489573, GM03:40489574, GM03:40557669, GM03:40591130, GM03:40703866, GM03:40554209, GM03:40589164, GM03:40606905, GM03:40632077, GM03:40705541, GM03:40576921, S06578-1-A, S16256-001-Q001, S16255-001-Q001, S16254-001-Q001, S16253-001-Q001, S16252-001-Q001, S16232-001-Q001 or a marker closely linked thereto.
   17. The isolated polynucleotide of embodiment 16, wherein the polynucleotide comprises
(a) a polynucleotide comprising SEQ ID NOs: 1, 2, 3, 4, 5, 6, 7, 8, 9, 10, 11, 12, 21 or 22;
(b) a polynucleotide comprising SEQ ID NOs: 23, 24, 25, 26, 27, 28, 29, 30, 31, 32, 33, 34, 45 or 46;
(c) a polynucleotide having at least 90% sequence identity to the polynucleotides set forth in parts (a) or (b); or
(d) a polynucleotide comprising at least 10 contiguous nucleotides of the polynucleotides set forth in parts (a) or (b).
   18. A kit for detecting or selecting at least one soybean plant or soybean germplasm with tolerance to chloride salt stress, the kit comprising:
a) primers or probes for detecting one or more marker loci associated with chloride salt stress tolerance, wherein the primers or probes are capable of detecting a marker locus comprising GM03:40563114, GM03:40576895, GM03:40489573, GM03:40489574, GM03:40557669, GM03:40591130, GM03:40703866, GM03:40554209, GM03:40589164, GM03:40606905, GM03:40632077, GM03:40705541, GM03:40576921, S06578-1-A, S16256-001-Q001, S16255-001-Q001, S16254-001-Q001, S16253-001-Q001, S16252-001-Q001, S16232-001-Q001 or a marker closely linked thereto; and
b) instructions for using the primers or probes for detecting the one or more marker loci and correlating the detected marker loci with predicted tolerance to chloride salt stress.
   19. A method of identifying a first soybean plant or a first soybean germplasm that displays tolerance to chloride salt stress, the method comprising detecting in the genome of said first soybean plant or in the genome of said first soybean germplasm at least one marker locus that is associated with the tolerance, wherein the marker locus is between about marker S04733-1-A and about marker S16227-001-K001 on linkage group N.
   20. The method of embodiments 19, wherein the at least one marker locus comprises S04733-1-A, S0045-1-A, S12869-1-Q1, S16226-001-K001, S16227-001-K001 or a marker closely linked thereto.
   21. The method of embodiment 19, wherein at least two or more marker loci are detected.
   22. The method of any one of embodiments 19-21, wherein the germplasm is a soybean variety.
   23. The method of any one of embodiments 19-22, wherein the method further comprises selecting the first soybean plant or first soybean germplasm or a progeny thereof having the at least one marker locus.
   24. The method of embodiment 23, further comprising crossing the selected first soybean plant or first soybean germplasm with a second soybean plant or second soybean germplasm.
   25. The method of embodiment 24, wherein the second soybean plant or second soybean germplasm comprises an exotic soybean strain or an elite soybean strain.
   26. The method of any one of embodiments 19-25, wherein the detecting comprises DNA sequencing of at least one of said marker loci.
   27. The method of any one of embodiments 19-25, wherein the detecting comprises amplifying at least one of said marker loci and detecting the resulting amplified marker amplicon.
   28. The method of embodiment 27, wherein the amplifying comprises:
a) admixing an amplification primer or amplification primer pair for each marker locus being amplified with a nucleic acid isolated from the first soybean plant or the first soybean germplasm, wherein the primer or primer pair is complementary or partially complementary to a variant or fragment of the genomic region comprising the marker locus, and is capable of initiating DNA polymerization by a DNA polymerase using the soybean nucleic acid as a template; and
b) extending the primer or primer pair in a DNA polymerization reaction comprising a DNA polymerase and a template nucleic acid to generate at least one amplicon.
   29. The method of embodiment 28, wherein said method comprises amplifying a variant or fragment of one or more polynucleotides comprising SEQ ID NOs: 53, 54, 55, 56 or 57.
   30. The method of embodiment 28, wherein said primer or primer pair comprises a variant or fragment of one or more polynucleotides comprising SEQ ID NOs: 53, 54, 55, 56, 57 or complements thereof.
   31. The method of embodiment 30, wherein said primer or primer pair comprises a nucleic acid sequence comprising SEQ ID NOs: 13, 14, 15, 16, 17, 18, 19, 20 or variants or fragments thereof.
   32. The method of embodiment 28, wherein the method further comprises providing one or more labeled nucleic acid probes suitable for detection of each marker locus being amplified.
   33. The method of embodiment 32, wherein said labeled nucleic acid probe comprises a nucleic acid sequence comprising a variant or fragment of one or more polynucleotides comprising SEQ ID NOs: 53, 54, 55, 56, 57 or complements thereof.
   34. The method of embodiment 33, wherein the labeled nucleic acid probe comprises a nucleic acid sequence comprising SEQ ID NOs: 35, 36, 37, 38, 39, 40, 41, 42, 43 or 44.
   35. An isolated polynucleotide capable of detecting a marker locus of the soybean genome comprising S12869-1-Q1, S00145-1-A, S16226-001-K001, S16227-001-K001, S04733-1-A or a marker closely linked thereto.
   36. The isolated polynucleotide of embodiment 35, wherein the polynucleotide comprises
(a) a polynucleotide comprising SEQ ID NOs: 13, 14, 15, 16, 17, 18, 19 or 20;
(b) a polynucleotide comprising SEQ ID NOs: 35, 36, 37, 38, 39, 40, 41, 42, 43 or 44;
(c) a polynucleotide having at least 90% sequence identity to the polynucleotides set forth in parts (a) or (b); or
(d) a polynucleotide comprising at least 10 contiguous nucleotides of the polynucleotides set forth in parts (a) or (b).
   37. A kit for detecting or selecting at least one soybean plant or soybean germplasm with tolerance to chloride salt stress, the kit comprising:
a) primers or probes for detecting one or more marker loci associated with chloride salt stress tolerance, wherein the primers or probes are capable of detecting a marker locus comprising S12869-1-Q1, S00145-1-A, S16226-001-K001, S16227-001-K001, S04733-1-A or a marker closely linked thereto; and
b) instructions for using the primers or probes for detecting the one or more marker loci and correlating the detected marker loci with predicted tolerance to chloride salt stress.

### EXPERIMENTAL

The following examples are offered to illustrate, but not to limit the claimed invention. It is understood that the examples and embodiments described herein are for illustrative purposes only, and persons skilled in the art will recognize various reagents or parameters that can be altered without departing from the spirit of the invention or the scope of the appended claims.

### Example 1

### Chloride Mapping in the Populations 95Y10×95Y40 and 95Y10×95Y70

Two F3 populations were employed in QTL analysis for chloride tolerance, 95Y10×95Y40 (Pop1) and 95Y10×95Y70 (Pop2). Lee et al., previously mapped chloride tolerance in salt-tolerant soybean cultivar S-100 near Sat_{—}091 on Lg-N, explaining up to 79% of the phenotypic variation (Lee et al. (2004) “A major QTL conditioning salt tolerance in S-100 soybean and descendent cultivars” Theor. Appl. Genet. 109:1610-19). The QTL on LG N was confirmed by composite interval mapping (CIM) in the population 95Y10×95Y40, which accounted for up to 69% of the phenotypic variation, and by single marker analysis (SMA) in the population 95Y10×95Y70, accounting for up to 53% of the variation. The effects came from 95Y40 and 95Y70.

The regions of significance among the two populations are summarized in Table 4.

TABLE 4
Chloride Salt Stress Tolerance QTL from Mapping Studies

| LG (ch) | Marker | Position (cM) | R2 | Analysis | Population | Phenotype Data |
|---|---|---|---|---|---|---|
| N (3) | S06578-1-Q2 | 61.61 | 0.649 | SMA | Pop1 | Abv % Chloride |
| N (3) | S06578-1-Q2 | 61.61 | 0.665 | SMA | Pop1 | Chloride Lab Score |
| N (3) | S06578-1-Q2 | 61.61 | 0.278 | SMA | Pop1 | Chloride Field Score |
| N (3) | S12869-1-Q1 | 54.91 | 0.653 | CIM | Pop1 | Abv % Chloride |
| N (3) | S12869-1-Q1 | 54.91 | 0.691 | CIM | Pop1 | Chloride Lab Score |
| N (3) | S12869-1-Q1 | 54.91 | 0.284 | CIM | Pop1 | Chloride Field Score |
| N (3) | S12869-1-Q1 | 54.91 | 0.663 | MIM | Pop1 | Abv % Chloride |
| N (3) | S12869-1-Q1 | 54.91 | 0.709 | MIM | Pop1 | Chloride Lab Score |
| N (3) | S12869-1-Q1 | 54.91 | 0.321 | MIM | Pop1 | Chloride Field Score |
| N (3) | S00145-1-A | 59.27 | 0.529 | SMA | Pop2 | Abv % Chloride |
| N (3) | S00145-1-A | 59.27 | 0.459 | SMA | Pop2 | Chloride Lab Score |
| N (3) | S00145-1-A | 59.27 | 0.346 | SMA | Pop2 | Chloride Field Score |

### Materials and Methods:

### Populations:

The F3 populations 95Y10×95Y40 and 95Y10×95Y70 consisting of 180 progeny each were submitted for genotyping. Genomic DNA was extracted from calluses or leaves using a modification of the CTAB (cetyltriethylammonium bromide, Sigma H5882) method described by Stacey and Isaac (Methods in Molecular Biology, Vol. 28: Protocols for Nucleic Acid Analysis by Nonradioactive Probes, Ed: Isaac, Humana Press Inc, Totowa, N.J. 1994, Ch 2, pp. 9-15). Approximately 100-200 mg of frozen tissues is ground into powder in liquid nitrogen and homogenised in 1 ml of CTAB extraction buffer (2% CTAB, 0.02 M EDTA, 0.1 M Tris-Cl pH 8, 1.4 M NaCl, 25 mM DTT) for 30 min at 65° C. Homogenised samples are allowed to cool at room temperature for 15 min before a single protein extraction with approximately 1 ml 24:1 v/v chloroform:octanol is done. Samples are centrifuged for 7 min at 13,000 rpm and the upper layer of supernatant collected using wide-mouthed pipette tips. DNA is precipitated from the supernatant by incubation in 95% ethanol on ice for 1 h. DNA threads are spooled onto a glass hook, washed in 75% ethanol containing 0.2 M sodium acetate for 10 min, air-dried for 5 min and resuspended in TE buffer. Five μl RNAse A is added to the samples and incubated at 37° C. for 1 h.

### Genotyping:

Evenly distributed polymorphic markers were selected across all 20 chromosomes for each population, resulting in 186 markers for 95Y10×95Y40 and 214 markers for 95Y10×95Y70. Each polymorphic marker set was used to genotype the respective population for which it was selected.

### Phenotyping:

Three phenotypic scores were provided for each progeny of both populations for the categories: Abv. % Chloride, Chloride Lab Score, and Chloride Field Score. The Abv. % Chloride is the percentage of chloride physically measured in the plant (see Example 4), while the Chloride Lab Score applies a 1-9 numbering system to the Abv. % Chloride data. The Chloride Field Scores are the phenotypic scores from the field, ranging in value from 1 to 9.

### Linkage Analysis:

Map Manager QTX.b20 (Manly et al. (2001) Mammalian Genome 12:930-932) was used to construct the linkage maps with the following parameters:
1) Linkage Evaluation: Intercross
2) Search Criteria: P=1e⁻⁵
3) Map Function: Kosambi
4) Cross Type: Line Cross

### QTL Analysis:

Single marker analysis, composite interval mapping, and multiple interval mapping were executed using QTL Cartographer 2.5 (Wang et al. (2011) Windows QTL Cartographer 2.5; Dept. of Statistics, North Carolina State University, Raleigh, N.C. Available online at statgen.ncsu.edu/qtlcart/WQTLCart.htm). Chromosomes with more than two linked markers were investigated with CIM. The standard CIM model and forward and backward regression method was used, and the likelihood ratio statistic (LRS) threshold for statistical significance to declare QTLs was determined by a 500 permutation test. The initial MIM model was determined using the MIM forward search method. The default criteria were used to add QTL and interactions to the model iteratively until a stable model was found.

### Marker Positions:

The genetic map positions for the markers provided herein are reported from the public genetic map at www.soybase.org (see also Choi et al. (2007) “A Soybean Transcript Map: Gene Distribution, Haplotype and Single-Nucleotide Polymorphism Analysis” Genetics 176:685-96, and Hyten, et al. (2010) “A High Density Integrated Genetic Linkage Map of Soybean and the Development of a 1536 Universal Soy Linkage Panel for Quantitative Trait Locus Mapping” Crop Science 50:960-968). The physical map positions for the markers are reported from the public physical map at www.phytozyome.net/soybean (see also Schmutz, J, et al. (2010) “Genome Sequence of the Palaeopolyploid Soybean” Nature 463:178-183.).

### Results:

### Genotyping:

The allele calls were converted to the A (maternal), B (paternal), H (heterozygous) convention for mapping analysis. Upon preliminary analysis of population 95Y10×95Y40 (Pop1), 17 markers were removed from the analysis for returning more than 30% missing data, and one marker was removed due to monomorphic parental calls. Eighteen markers showed severe segregation distortion (p<0.0001) but were retained in the analysis. 14 progeny were also removed from the analysis due to missing data in excess of 30%. In the population 95Y10×95Y70 (Pop2), 11 markers and 36 progeny were identified as missing more than 30% data, 6 markers returned monomorphic parental calls and one returned monomorphic progeny calls, and 27 markers were severely distorted.

### Phenotyping:

The phenotypic distributions for each population indicated that the population was segregating for the trait of interest.

### Mapping Analysis:

The linkage maps were constructed using non-distorted markers to create a framework, and distorted markers were then distributed into the linkage groups where possible. Marker order was checked against a reference genetic map to ensure distorted markers distributed to the correct locations. For population 95Y10×95Y40, 137 markers formed 44 linkage groups. Five distorted markers were successfully distributed, while 26 markers remained unlinked. For population 95Y10×95Y70, 179 markers formed 43 linkage groups, and 17 markers remained unlinked. The linkage map and cross data for each population was exported in QTL Cartographer format for subsequent analysis.

### 95Y10×95Y40 QTL Analyses:

### Single Marker Analysis: QTL on Lg-N

Single marker analysis indicated a QTL on Lg-N at marker 506578-1-Q2 (61.61 cM) for all three data sets, explaining 64.9%, 66.5%, and 27.8% of the phenotypic variation.

### Composite Interval Mapping: QTL on Lg-N

A QTL was indicated by composite interval mapping on Lg-N for all three data sets, explaining 65.3%, 69.1%, and 28.4% of the phenotypic variation. The QTL effect was from 95Y40. The composite interval mapping results for the three data sets are shown in Table 4.

### Multiple Interval Mapping: Lg-N

Multiple interval mapping confirmed the QTL on Lg-N with percent variation explained ranging from 32.1% to 70.9%.

### 95Y10×95Y70 QTL Analysis:

### Single Marker Analysis: QTL Lg-N

A QTL was found by single marker analysis on Lg-N at marker S00145-1-A (59.27 cM) for all three data sets. The percent variation explained was 52.9%, 45.9%, and 34.6%.

### Multiple Interval Mapping:

No QTLs were significant by multiple interval mapping in the population 95Y10×95Y70.

### Example 2

### Fine Mapping a Chloride QTL on Lg-N

Several QTLs were identified using the two mapping populations as described in Example 1. Further work to examine the QTL on LG N was initiated. An association study further defined the QTL interval, and SNPs were identified that perfectly differentiated lines that were susceptible and tolerant to chloride. TaqMan™ markers were designed at these SNPs and additional KASPar markers were created to saturate the QTL region. This analysis combines the genotypic information from the initial study described in Example 1 with data from the new markers to fine map the chloride QTL on LG N. A QTL was identified in each population for all three phenotype data sets with peaks between about 54.91 cM and 61.78 cM on LG N (GM03), explaining up to 71% of the phenotypic variation. Marker S16232-001-Q001 (61.51 cM) was the most consistent peak marker across populations and data sets using single marker analysis. Composite interval mapping indicated the peak TaqMan™ marker between about 60.6 cM and 62.4 cM among the data sets.

### Materials and Methods:

### Population:

The F3 populations used and DNA preparation was done as described in Example 1.

### Genotyping:

From the polymorphic marker sets identified in Example 1, 10 markers were from Lg-N for population 95Y10×95Y40, and 11 markers were from Lg-N for 95Y10×95Y70. Eight TaqMan™ markers were designed using SNPs that perfectly differentiated between tolerant and susceptible lines and 31 additional KASPar markers were created to provide additional coverage across the QTL interval.

### Phenotyping:

Three phenotypic score data sets as described in Example 1 were provided for each progeny of both populations.

### QTL Analysis:

Genetic positions were calculated for new markers using the physical coordinates and known genetic positions for flanking markers on a genetic map. The data sets were then arranged by genetic position and import files were manually created for downstream analysis.

Single marker analysis and composite interval mapping were executed using QTL Cartographer 2.5 (Wang et al. (2011) Windows QTL Cartographer 2.5; Dept. of Statistics, North Carolina State University, Raleigh, N.C. Available online at statgen.ncsu.edu/qtlcart/WQTLCart.htm). The standard CIM model and forward and backward regression method was used, and the LRS threshold for statistical significance to declare QTLs was determined by a 500 permutation test.

### Results:

### Genotyping:

For population 95Y10×95Y40, 17 markers failed, 7 were missing greater than 30% data, and two were highly distorted (p<0.0001). 56 progeny were missing more than 30% data. For the population 95Y10×95Y70, 20 markers failed, one was highly distorted, and 49 progeny were missing more than 30% data. These markers and individuals were removed from subsequent analysis and the remaining allele calls were converted to the A (Maternal) B (Paternal) H (Heterozygous) convention for QTL analysis.

### Phenotyping:

The phenotypic distributions for each population are the same as those shown in Example 1.

### QTL Analysis 95Y10×95Y40:

### Single Marker Analysis:

Single marker analysis indicated significant markers across all three data sets with peak markers located between about 60.94 cM and 61.54 cM.

### Composite Interval Mapping: QTL on Lg-N

Significant QTLs were found on Lg-N using each of the three phenotype data sets, explaining up to 71% of the phenotypic variation. Abv. % Chloride showed two peaks at about 54 cM and 62 cM. The Lab Score and Field Score data sets showed one peak around about 60 to 61 cM. These results are summarized in Table 5.

TABLE 5
QTL Analysis 95Y10 × 95Y40

| | Position | Abv % Cl | | | Cl Lab | | | Cl Field | | |
|---|---|---|---|---|---|---|---|---|---|---|
| Marker | (cM) | LRS | Pr(F) | R2 | LRS | Pr(F) | R2 | LRS | Pr(F) | R2 |
| S12869 | 54.91 | 22.670 | 0.00000 | 0.164 | 71.898 | 0.00000 | 0.431 | 16.802 | 0.00005 | 0.125 |
| S16226 | 60.59 | 49.270 | 0.00000 | 0.293 | 150.381 | 0.00000 | 0.657 | 27.007 | 0.00000 | 0.172 |
| S16227 | 60.59 | 37.365 | 0.00000 | 0.227 | 143.755 | 0.00000 | 0.629 | 32.504 | 0.00000 | 0.191 |
| S06578 | 61.61 | 50.521 | 0.00000 | 0.326 | 157.107 | 0.00000 | 0.703 | 29.050 | 0.00000 | 0.204 |
| S16256 | 61.55 | 50.433 | 0.00000 | 0.308 | 156.419 | 0.00000 | 0.661 | 29.838 | 0.00000 | 0.199 |
| S16255 | 61.65 | 47.532 | 0.00000 | 0.314 | 147.410 | 0.00000 | 0.690 | 28.358 | 0.00000 | 0.202 |
| S16254 | 61.45 | 37.719 | 0.00000 | 0.207 | 150.573 | 0.00000 | 0.577 | 36.105 | 0.00000 | 0.191 |
| S16252 | 61.45 | 49.038 | 0.00000 | 0.313 | 152.759 | 0.00000 | 0.674 | 29.394 | 0.00000 | 0.198 |
| S16232 | 61.51 | 50.771 | 0.00000 | 0.332 | 157.700 | 0.00000 | 0.714 | 28.184 | 0.00000 | 0.200 |
| S04733 | 44.70 | 11.443 | 0.00081 | 0.080 | 26.840 | 0.00000 | 0.179 | 4.731 | 0.03128 | 0.034 |

### QTL Analysis 95Y10×95Y70:

### Single Marker Analysis:

Single marker analysis indicated significant markers across all three data sets with peak markers located between about 61.45 cM and 61.51 cM on LG N. These results are summarized in Table 6.

TABLE 6
QTL Analysis 95Y10 × 95Y70

| | Position | Abv % Cl | | | Cl Lab | | | Cl Field | | |
|---|---|---|---|---|---|---|---|---|---|---|
| Marker | (cM) | LRS | Pr(F) | R2 | LRS | Pr(F) | R2 | LRS | Pr(F) | R2 |
| S16226 | 60.59 | 116.591 | 0.00000 | 0.589 | 90.593 | 0.00000 | 0.499 | 58.110 | 0.00000 | 0.358 |
| S16227 | 60.59 | 107.021 | 0.00000 | 0.576 | 85.559 | 0.00000 | 0.494 | 51.190 | 0.00000 | 0.333 |
| S16256 | 61.55 | 138.158 | 0.00000 | 0.631 | 109.771 | 0.00000 | 0.544 | 61.116 | 0.00000 | 0.346 |
| S16255 | 61.65 | 110.385 | 0.00000 | 0.555 | 87.724 | 0.00000 | 0.472 | 53.151 | 0.00000 | 0.323 |
| S16254 | 61.45 | 123.283 | 0.00000 | 0.595 | 96.806 | 0.00000 | 0.506 | 59.467 | 0.00000 | 0.326 |
| S16252 | 61.45 | 116.211 | 0.00000 | 0.567 | 91.778 | 0.00000 | 0.479 | 50.979 | 0.00000 | 0.294 |
| S16232 | 61.51 | 130.832 | 0.00000 | 0.632 | 105.031 | 0.00000 | 0.552 | 61.853 | 0.00000 | 0.376 |
| S04733 | 44.70 | 9.508 | 0.00226 | 0.057 | 10.007 | 0.00173 | 0.060 | 8.976 | 0.00300 | 0.061 |
| S00145 | 59.27 | 127.504 | 0.00000 | 0.591 | 99.211 | 0.00000 | 0.524 | 62.836 | 0.00000 | 0.373 |

### Composite Interval Mapping: QTL on Lg-N

All three phenotype data sets showed significant QTLs on Lg-N, explaining up to 70% of the phenotypic variation. Abv. % Chloride and Field Score data sets showed two peaks; one between about 57.6 and 58.6 cM, and the other between about 61.5 cM and 62 cM. The Lab Score data set showed one peak around 61.5 cM.

### Example 3

### Case Control Association Analysis: Chloride Salt Stress Tolerance

Using a case-control association analysis, a previously identified QTL conditioning variation in chloride salt stress was putatively fine-mapped between 40454221-40759329 bp on Gm03 (Lg N). A set of 13 SNPs were identified in this region that perfectly differentiate highly tolerate from susceptible lines. These markers are ideal for marker-assisted selection of chloride salt stress tolerance.

### Methods:

DNA was prepped using standard Illumina TruSeq Chemistry and lines were sequenced on an Illumina HiSeq2000. SNPs were called using a proprietary sequence analysis software. Haploview (Barrett et al. (2005) Bioinformatics 21:263-265) was used to conduct a case-control association analysis on a set of 9870 SNPs identified in the region from 37622605-41590045 bp on Gm03. The case group comprised 21 proprietary soybean lines susceptible to chloride salt stress and the control group comprised 12 public and proprietary lines tolerant to chloride salt stress.

### Results and Discussion:

Chi square values from case-control analysis vs. physical position of 9870 SNPs revealed a peak of SNP-to-trait association between 40454221-40759329 bp on Gm03, suggesting that a locus conditioning salt tolerance is present in this region.
Table 7 shows 13 SNPs that were identified having a perfect association between 21 susceptible (case) and 12 tolerant (control) lines. These markers are ideal targets for TaqMan™, or other comparable detection method, assay design.

TABLE 7

| | | Assoc | Case, Control | Case, Control | Chi | |
|---|---|---|---|---|---|---|
| Name | Phys Pos | Allele | Ratio Counts | Frequencies | square | P value |
| Gm03:40563114 | 40563114 | T | 42:0, 0:24 | 1.000, 0.000 | 66 | 4.51E−16 |
| Gm03:40576895 | 40576895 | A | 42:0, 0:24 | 1.000, 0.000 | 66 | 4.51E−16 |
| Gm03:40489573 | 40489573 | C | 42:0, 0:22 | 1.000, 0.000 | 64 | 1.24E−15 |
| Gm03:40489574 | 40489574 | T | 42:0, 0:22 | 1.000, 0.000 | 64 | 1.24E−15 |
| Gm03:40557669 | 40557669 | A | 42:0, 0:22 | 1.000, 0.000 | 64 | 1.24E−15 |
| Gm03:40591130 | 40591130 | T | 40:0, 0:24 | 1.000, 0.000 | 64 | 1.24E−15 |
| Gm03:40703866 | 40703866 | T | 40:0, 0:24 | 1.000, 0.000 | 64 | 1.24E−15 |
| Gm03:40554209 | 40554209 | T | 40:0, 0:22 | 1.000, 0.000 | 62 | 3.43E−15 |
| Gm03:40589164 | 40589164 | T | 40:0, 0:22 | 1.000, 0.000 | 62 | 3.43E−15 |
| Gm03:40606905 | 40606905 | T | 40:0, 0:22 | 1.000, 0.000 | 62 | 3.43E−15 |
| Gm03:40632077 | 40632077 | C | 42:0, 0:20 | 1.000, 0.000 | 62 | 3.43E−15 |
| Gm03:40705541 | 40705541 | A | 40:0, 0:22 | 1.000, 0.000 | 62 | 3.43E−15 |
| Gm03:40576921 | 40576921 | C | 42:0, 0:18 | 1.000, 0.000 | 60 | 9.49E−15 |

Table 8 lists the allele calls at the 13 markers with a perfect association to tolerant or susceptible phenotypes. Chloride scores range from susceptible, 1 to tolerant, 9. Boxes with “.” represent missing data and lower case letters represent imputed allele calls.

TABLE 8

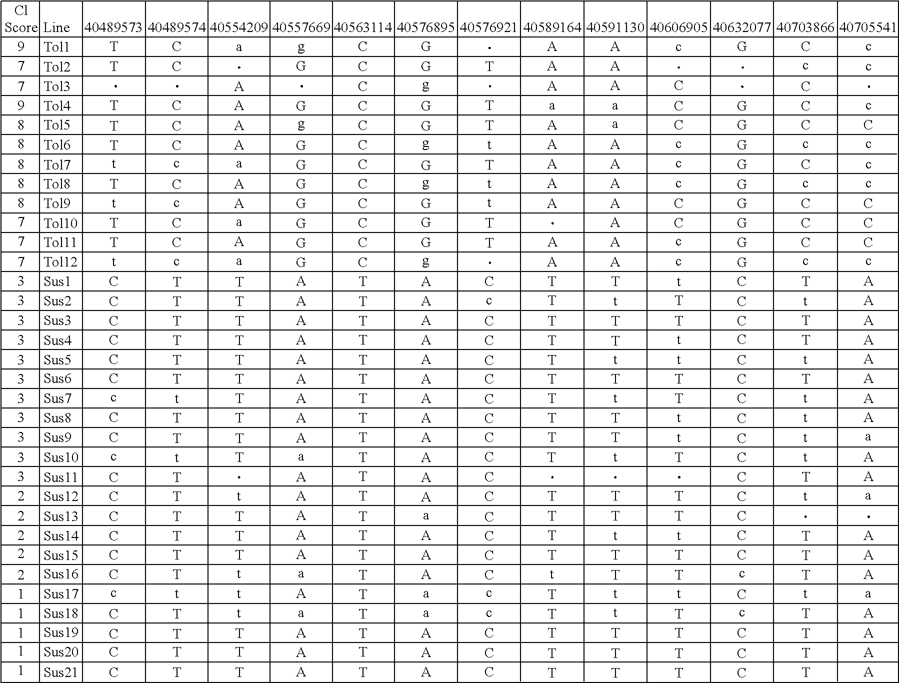

Table 9 lists the map positions, and SNP allele calls for several marker loci associated with chloride salt stress tolerance on LG N (chromosome 3).

TABLE 9

| | Genetic | Physical | |
|---|---|---|---|
| Marker Name | Position | Position | Allele |
| S12869-1-Q1 | 54.91 | 38,806,917 | A/G |
| S00145-1-A | 59.27 | 39,796,244 | A/C |
| S16226-001-K001 | 60.59 | 40,061,119 | T/C |
| S16227-001-K001 | 60.59 | 40 061,269 | G/A |
| S04733-1-A | 44.70 | 37,017,850 | T/C |
| S06578-1-A | 61.61 | 40,672,157 | C/G |
| S16256 | 61.55 | 40,632,077 | C/G |
| S16255 | 61.65 | 40,703,866 | T/C |
| S16254 | 61.45 | 40,557,669 | A/G |
| S16253 | 61.47 | 40,576,895 | A/G |
| S16252 | 61.45 | 40,563,114 | T/C |
| S16232 | 61.51 | 40,703,866 | T/C |

Tables 1, 2 and 3 list SNP markers and provide TaqMan™ primers, probes and sequences that can be used for identifying and/or detecting the SNP markers associated with tolerance to chloride salt stress.

One hundred twenty-six public and proprietary soybean lines were screened to characterize their haplotype as defined by the 13 markers and haplotypes in Tables 7 and 8. From this screen, 30 of the public and proprietary varieties showed the tolerant haplotype, and 96 of the public and proprietary varieties showed the susceptible phenotype. For most of these lines however their phenotypic score has not yet been validated. The panel did include known excluder chloride tolerant line Lee, and known accumulator lines Jackson and Essex, and the haplotypes were consistent with their known phenotypes. Based on the marker haplotypes, it is expected that chloride salt tolerance phenotype data will confirm each line's assignment to tolerant versus susceptible class.

### Example 4

### Chloride Screening and Analysis in Soybean

Seeds for soybean varieties to be screened were planted one seed/pot in 2″ D16 DEEPOTS™ placed into D50T trays (30 pots/tray). Seeds were planted in potting soil in a randomized experimental block design with 4 replications each. D50T trays with pots were places in Black Flood Trays (9 D50T/flood tray). The experimental design included seed from chloride excluder (tolerant) check variety Morgan, Lee, and Bedford, and chloride accumulators (susceptible) varieties Bragg, Jackson, Hutchinson and Essex used as reference varieties. Seeds germinated, emerged, and matured to the V2-V3 growth stage before chloride treatment. Chloride treatment consisted of a 14 day treatment period with 14.7014 g/L CaCl, or a water control. Plants are visually scored for symptoms of chloride toxicity 14-21 days after treatment using the following criteria:
9=healthy, no apparent symptoms of chlorosis
6=slight chlorosis, 25% of the leaf area shows chlorosis symptoms
3=severe chlorosis, 75% of the leaf area shows chlorosis symptoms
1=dead, plants are brown and withered.

Plant leaf tissue was collected for chloride analysis by inductively coupled plasma spectroscopy (ICP). Samples of recently mature trifoliate leaves were taken from the top of each plant to be tested. The plant material was air dried in the shade, ground to a powder, and passed through a 1.0 mm screen. Approximately 100 mg of prepared tissue is used for chloride analysis. Care is taken to avoid contamination with exogenous chloride from metal containers or implements.

Chloride analysis by ICP was done by the University of Arkansas Diagnostic Lab, essentially as described in Wheal & Palmer (2010 J Anal At Spectrom 25:1946-1952), except instead of using 4% (v/v) nitric acid as describe in Wheal & Palmer, leaf samples were extracted using hot water extraction (Ghosh & Drew (1991) 136:265-268). As in Wheal & Palmer appropriate reference samples are included in each analysis.

Chloride analysis results were reported as mg chloride/kg. These results were converted to % chloride (w/w) and rounded to the nearest hundredth (Abv % Cl). These results were then evenly grouped on a 1-9 scale and used as one of the phenotype data sets in the mapping studies described in Examples 1-3.

TABLE 10
Summary of SEQ ID NOs.

| SEQ ID NO | Description |
|---|---|
| 1 | Primer S16256-F001 |
| 2 | Primer S16256-R001 |
| 3 | Primer S16255-F001 |
| 4 | Primer S16255-R001 |
| 5 | Primer S16254-F001 |
| 6 | Primer S16254-R001 |
| 7 | Primer S16253-F001 |
| 8 | Primer S16253-R001 |
| 9 | Primer S16252-F001 |
| 10 | Primer S16252-R001 |
| 11 | Primer S16232-F001 |
| 12 | Primer S16232-R001 |
| 13 | Primer S04733-F001 |
| 14 | Primer S04733-R001 |
| 15 | Primer S00145-F001 |
| 16 | Primer S00145-R001 |
| 17 | Primer S16227-R001 |
| 18 | Primer S12869-F001 |
| 19 | Primer S12869-R001 |
| 20 | Primer S16226-R001 |
| 21 | Primer S06578-F001 |
| 22 | Primer S06578-R001 |
| 23 | Probe S16256-001-X001 |
| 24 | Probe S16256-001-X002 |
| 25 | Probe S16255-001-X001 |
| 26 | Probe S16255-001-X002 |
| 27 | Probe S16254-001-X001 |
| 28 | Probe S16254-001-X002 |
| 29 | Probe S16253-001-X001 |
| 30 | Probe S16253-001-X002 |
| 31 | Probe S16252-001-X001 |
| 32 | Probe S16252-001-X002 |
| 33 | Probe S16232-001-X003 |
| 34 | Probe S16232-001-X004 |
| 35 | Probe S04733-1-P1 |
| 36 | Probe S04733-1-P2 |
| 37 | Probe S00145-1-P1 |
| 38 | Probe S00145-1-P2 |
| 39 | Probe S16227-001-P1 |
| 40 | Probe S16227-001-P2 |
| 41 | Probe S12869-1-P1 |
| 42 | Probe S12869-1-P2 |
| 43 | Probe S16226-001-P1 |
| 44 | Probe S16226-001-P2 |
| 45 | Probe S06578-1-P1 |
| 46 | Probe S06578-1-P2 |
| 47 | Design Sequence gmchr3v1: 40632077_201 |
| 48 | Design Sequence gmchr3v1: 40557669_201 |
| 49 | Design Sequence gmchr3v1: 40703866_201 |
| 50 | Design Sequence gmchr3v1: 40563114_201 |
| 51 | Design Sequence gmchr3v1: 40576895_201 |
| 52 | Design Sequence gmchr3v1: 40606905_201 |
| 53 | Design Sequence S04733-1 |
| 54 | Design Sequence S00145-1 |
| 55 | Design Sequence gmchr3v1: 400061269-201 |
| 56 | Design Sequence S12869-1 |
| 57 | Design Sequence gmchr3v1: 40061119-201 |
| 58 | Design Sequence S06578-1 |

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

That which is claimed:

1. A method of identifying a first soybean plant or a first soybean germplasm that displays tolerance to chloride salt stress, the method comprising detecting in the genome of said first soybean plant or in the genome of said first soybean germplasm at least one marker locus that is associated with the tolerance, wherein the at least one marker locus comprises
(a) GM03:40563114, GM03:40576895, GM03:40489573, GM03:40489574, GM03:40557669, GM03:40591130, GM03:40703866, GM03:40554209, GM03:40589164, GM03:40606905, GM03:40632077, GM03:40705541, GM03:40576921, S06578-1-A, S16256-001-Q001, S16255-001-Q001, S16254-001-Q001, S16253-001-Q001, S16252-001-Q001, S16232-001-Q001 or a marker closely linked thereto;
(b) a marker locus between about marker S04733-1-A and about marker S16227-001-K001 on linkage group N; or
(c) S04733-1-A, S0045-1-A, S12869-1-Q1, S16226-001-K001, S16227-001-K001 or a marker closely linked thereto.

2. The method of claim 1, wherein at least two or more of the marker loci are detected.

3. The method of claim 1, wherein the germplasm is a soybean variety.

4. The method of claim 1, wherein the method further comprises selecting the first soybean plant or first soybean germplasm or a progeny thereof having the at least one marker locus.

5. The method of claim 4, further comprising crossing the selected first soybean plant or first soybean germplasm with a second soybean plant or second soybean germplasm.

6. The method of claim 5, wherein the second soybean plant or second soybean germplasm comprises an exotic soybean strain or an elite soybean strain.

7. The method of claim 1, wherein the detecting comprises DNA sequencing of at least one of said marker loci.

8. The method of claim 1, wherein the detecting comprises amplifying at least one of said marker loci and detecting the resulting amplified marker amplicon.

9. The method of claim 8, wherein the amplifying comprises:
a) admixing an amplification primer or amplification primer pair for each marker locus being amplified with a nucleic acid isolated from the first soybean plant or the first soybean germplasm, wherein the primer or primer pair is complementary or partially complementary to a variant or fragment of the genomic region comprising the marker locus, and is capable of initiating DNA polymerization by a DNA polymerase using the soybean nucleic acid as a template; and
b) extending the primer or primer pair in a DNA polymerization reaction comprising a DNA polymerase and a template nucleic acid to generate at least one amplicon.

10. The method of claim 9, wherein said method comprises
(a) amplifying a variant or fragment of one or more polynucleotides comprising SEQ ID NOs: 47, 48, 49, 50, 51, 52 or 58; or
(b) amplifying a variant or fragment of one or more polynucleotides comprising SEQ ID NOs: 53, 54, 55, 56 or 57.

11. The method of claim 9, wherein said primer or primer pair comprises
(a) a variant or fragment of one or more polynucleotides comprising SEQ ID NOs: 47, 48, 49, 50, 51, 52, 58 or complements thereof; or
(b) a variant or fragment of one or more polynucleotides comprising SEQ ID NOs: 53, 54, 55, 56, 57 or complements thereof.

12. The method of claim 11, wherein said primer or primer pair comprises
(a) a nucleic acid sequence comprising SEQ ID NOs: 1, 2, 3, 4, 5, 6, 7, 8, 9, 10, 11, 12, 21, 22 or variants or fragments thereof; or
(b) a nucleic acid sequence comprising SEQ ID NOs: 13, 14, 15, 16, 17, 18, 19, 20 or variants or fragments thereof.

13. The method of claim 9, wherein the method further comprises providing one or more labeled nucleic acid probes suitable for detection of each marker locus being amplified.

14. The method of claim 13, wherein said labeled nucleic acid probe comprises
(a) a nucleic acid sequence comprising a variant or fragment of one or more polynucleotides comprising SEQ ID NOs: 47, 48, 49, 50, 51, 52, 58 or complements thereof; or
(b) a nucleic acid sequence comprising a variant or fragment of one or more polynucleotides comprising SEQ ID NOs: 53, 54, 55, 56, 57 or complements thereof.

15. The method of claim 14, wherein the labeled nucleic acid probe comprises
(a) a nucleic acid sequence comprising SEQ ID NOs: 23, 24, 25, 26, 27, 28, 29, 30, 31, 32, 33, 34, 45 or 46; or
(b) a nucleic acid sequence comprising SEQ ID NOs: 35, 36, 37, 38, 39, 40, 41, 42, 43 or 44.

16. An isolated polynucleotide capable of detecting a marker locus of the soybean genome comprising (a) GM03:40563114, GM03:40576895, GM03:40489573, GM03:40489574, GM03:40557669, GM03:40591130, GM03:40703866, GM03:40554209, GM03:40589164, GM03:40606905, GM03:40632077, GM03:40705541, GM03:40576921, S06578-1-A, S16256-001-Q001, S16255-001-Q001, S16254-001-Q001, S16253-001-Q001, S16252-001-Q001, S16232-001-Q001 or a marker closely linked thereto; or
(b) S12869-1-Q1, S00145-1-A, S16226-001-K001, S16227-001-K001, S04733-1-A or a marker closely linked thereto.

17. The isolated polynucleotide of claim 16, wherein the polynucleotide comprises
(a) a polynucleotide comprising
(i) SEQ ID NOs: 1, 2, 3, 4, 5, 6, 7, 8, 9, 10, 11, 12, 21 or 22; or
(ii) SEQ ID NOs: 13, 14, 15, 16, 17, 18, 19 or 20;
(b) a polynucleotide comprising
(i) SEQ ID NOs: 23, 24, 25, 26, 27, 28, 29, 30, 31, 32, 33, 34, 45 or 46; or
(ii) SEQ ID NOs: 35, 36, 37, 38, 39, 40, 41, 42, 43 or 44;
(c) a polynucleotide having at least 90% sequence identity to the polynucleotides set forth in parts (a) or (b); or
(d) a polynucleotide comprising at least 10 contiguous nucleotides of the polynucleotides set forth in parts (a) or (b).

18. A kit for detecting or selecting at least one soybean plant or soybean germplasm with tolerance to chloride salt stress, the kit comprising:
a) primers or probes for detecting one or more marker loci associated with chloride salt stress tolerance, wherein the primers or probes are capable of detecting a marker locus comprising
(i) GM03:40563114, GM03:40576895, GM03:40489573, GM03:40489574, GM03:40557669, GM03:40591130, GM03:40703866, GM03:40554209, GM03:40589164, GM03:40606905, GM03:40632077, GM03:40705541, GM03:40576921, S06578-1-A, S16256-001-Q001, S16255-001-Q001, S16254-001-Q001, S16253-001-Q001, S16252-001-Q001, S16232-001-Q001 or a marker closely linked thereto; or
(ii) S12869-1-Q1, S00145-1-A, S16226-001-K001, S16227-001-K001, S04733-1-A or a marker closely linked thereto; and
b) instructions for using the primers or probes for detecting the one or more marker loci and correlating the detected marker loci with predicted tolerance to chloride salt stress.


SEQUENCE LISTING (58 sequences)

SEQ ID NO: 1 - DNA, 29 bp, Artificial Sequence
  Primer S16256-F001
atgctttacattgctattgatgtagtagt

SEQ ID NO: 2 - DNA, 30 bp, Artificial Sequence
  Primer S16256-R001
cgtagttgacaagttagttaaaaggtaaaa

SEQ ID NO: 3 - DNA, 27 bp, Artificial Sequence
  Primer S16255-F001
aagcatgaattattggattttgttaat

SEQ ID NO: 4 - DNA, 21 bp, Artificial Sequence
  Primer S16255-R001
ggcaaatgctaatgttggtgt

SEQ ID NO: 5 - DNA, 26 bp, Artificial Sequence
  Primer S16254-F001
aagatcacacatatgagcaagtaggc

SEQ ID NO: 6 - DNA, 24 bp, Artificial Sequence
  Primer S16254-R001
tttggattttggagtatgaatgaa

SEQ ID NO: 7 - DNA, 30 bp, Artificial Sequence
  Primer S16253-F001
tgcgcattaaatatacattagaaattttag

SEQ ID NO: 8 - DNA, 26 bp, Artificial Sequence
  Primer S16253-R001
tccaattttaccctttattcttacga

SEQ ID NO: 9 - DNA, 18 bp, Artificial Sequence
  Primer S16252-F001
cagcaagaagccgcaagt

SEQ ID NO: 10 - DNA, 29 bp, Artificial Sequence
  Primer S16252-R001
ctcactcgagtaaaacactaccattatta

SEQ ID NO: 11 - DNA, 24 bp, Artificial Sequence
  Primer S16232-F001
gacgccaaatagaagcgatagtaa

SEQ ID NO: 12 - DNA, 24 bp, Artificial Sequence
  Primer S16232-R001
tgtgttagactgacgtgataacca

SEQ ID NO: 13 - DNA, 19 bp, Artificial Sequence
  Primer S04733-F001
cagttgccacaggagttgc

SEQ ID NO: 14 - DNA, 20 bp, Artificial Sequence
  Primer S04733-R001
gggctggataggttcttcaa

SEQ ID NO: 15 - DNA, 22 bp, Artificial Sequence
  Primer S00145-F001
gtgcaaaaagcaaaccctgtgg

SEQ ID NO: 16 - DNA, 27 bp, Artificial Sequence
  Primer S00145-R001
gcaaagagtgacacttaagcagtgcaa

SEQ ID NO: 17 - DNA, 29 bp, Artificial Sequence
  Primer S16227-R001
ggatatggaaagcaacaaactttctggta

SEQ ID NO: 18 - DNA, 20 bp, Artificial Sequence
  Primer S12869-F001
cccgtcatgaaccatacaca

SEQ ID NO: 19 - DNA, 20 bp, Artificial Sequence
  Primer S12869-R001
tctttcatgtttggcacagc

SEQ ID NO: 20 - DNA, 30 bp, Artificial Sequence
  Primer S16226-R001
ccaaagtccttgagaaattgttcatcacta

SEQ ID NO: 21 - DNA, 25 bp, Artificial Sequence
  Primer S06578-F001
caattttgaccaatatttccagttc

SEQ ID NO: 22 - DNA, 20 bp, Artificial Sequence
  Primer S06578-R001
gtggtgaaccttgtcgtgaa

SEQ ID NO: 23 - DNA, 17 bp, Artificial Sequence
  Probe S16256-001-X001
tcatctagttcaatggt

SEQ ID NO: 24 - DNA, 17 bp, Artificial Sequence
  Probe S16256-001-X002
tcatctagttgaatggt

SEQ ID NO: 25 - DNA, 17 bp, Artificial Sequence
  Probe S16255-001-X001
ttacacataccaataaa

SEQ ID NO: 26 - DNA, 19 bp, Artificial Sequence
  Probe S16255-001-X002
attttacacacaccaataa

SEQ ID NO: 27 - DNA, 15 bp, Artificial Sequence
  Probe S16254-001-X001
cggttggactattga

SEQ ID NO: 28 - DNA, 16 bp, Artificial Sequence
  Probe S16254-001-X002
cggttggactactgat

SEQ ID NO: 29 - DNA, 18 bp, Artificial Sequence
  Probe S16253-001-X001
cataattgactcattaac

SEQ ID NO: 30 - DNA, 19 bp, Artificial Sequence
  Probe S16253-001-X002
ataacataattggctcatt

SEQ ID NO: 31 - DNA, 16 bp, Artificial Sequence
  Probe S16252-001-X001
cataaaccaaaaactg

SEQ ID NO: 32 - DNA, 17 bp, Artificial Sequence
  Probe S16252-001-X002
aacataaaccagaaact

SEQ ID NO: 33 - DNA, 15 bp, Artificial Sequence
  Probe S16232-001-X003
acgacaccatattga

SEQ ID NO: 34 - DNA, 15 bp, Artificial Sequence
  Probe S16232-001-X004
acgacaccgtattga

SEQ ID NO: 35 - DNA, 15 bp, Artificial Sequence
  Probe S04733-1-P1
accattagaaactcg

SEQ ID NO: 36 - DNA, 15 bp, Artificial Sequence
  Probe S04733-1-P2
accattaggaactcg

SEQ ID NO: 37 - DNA, 17 bp, Artificial Sequence
  Probe S00145-1-P1
tgcaattctattaagcc

SEQ ID NO: 38 - DNA, 16 bp, Artificial Sequence
  Probe S00145-1-P2
caattctcttaagccc

SEQ ID NO: 39 - DNA, 46 bp, Artificial Sequence
  Probe S16227-001-P1
gaaggtgaccaagttcatgctaattgactcaacttacttacctcag

SEQ ID NO: 40 - DNA, 49 bp, Artificial Sequence
  Probe S16227-001-P2
gaaggtcggagtcaacggattcctaattgactcaacttacttacctcaa

SEQ ID NO: 41 - DNA, 18 bp, Artificial Sequence
  Probe S12869-1-P1
ttttaaccatgcttttgg

SEQ ID NO: 42 - DNA, 16 bp, Artificial Sequence
  Probe S12869-1-P2
tttagccatgcttttg

SEQ ID NO: 43 - DNA, 48 bp, Artificial Sequence
  Probe S16226-001-P1
gaaggtgaccaagttcatgctatctgcgatgtttccagttaactgatt

SEQ ID NO: 44 - DNA, 46 bp, Artificial Sequence
  Probe S16226-001-P2
gaaggtcggagtcaacggattctgcgatgtttccagttaactgatc

SEQ ID NO: 45 - DNA, 15 bp, Artificial Sequence
  Probe S06578-1-P1
ccaagctaacatgtc

SEQ ID NO: 46 - DNA, 15 bp, Artificial Sequence
  Probe S06578-1-P2
ccaagctaagatgtc

SEQ ID NO: 47 - DNA, 401 bp, Artificial Sequence
  Design Sequence gmchr3v140632077_201
aaatatttaccaacacatggtgccggtgagagaatccttgacgcttggagtgttaatgaacaccaaagggttggtggagctaatcgttctcaatattggcagagagaagaaggtgagttatgctttacattgctattgatgtagtagtaattatacaaatatgttgttttgtgttattctacgattatcatcatctagttcaatggttaactttatcacatatttatagttgaatataagttagtttattaattttcaactaaaaattaactttttttaccttttaactaacttgtcaactacgtatctttttgtcgtacacactatgaggtgtcacttattttcatagactcttatgagaatagtacatacacacacgaaatttaacttttttctttaaa

SEQ ID NO: 48 - DNA, 401 bp, Artificial Sequence
  Design Sequence gmchr3v140557669_201
cttaactaatatcctataaacactagttaacatttgccaataatttttacatgttattactttggtaaaatcattrtataactaagcatgaattattggattttgttaattagtgttttttgacattgattaagaaattaaaaggaaaaaatatttattatggagaggcataaaaaaaacaaataatataattttacacataccaataaaaatctttctctttttaattcctaaaccaatacaccaacattagcatttgccaaggttgtttgtctcaaaaaattatggaaagtaccttttgaaggtaacattcccttatccacaaatagcttagtcatgtcaaattgttgtactgccatccatatacatcatgccttaattagtccaagacacctcaatca

SEQ ID NO: 49 - DNA, 401 bp, Artificial Sequence
  Design Sequence gmchr3v140703866_201
acagagaaaattgaatataaacaagcagaactgtaaaataggaatcacttcaaaatagagtgcaattaggttcaaacaaatgtgaagttaccaatgaaatgcaaagtaagatcacacatatgagcaagtaggctcggggcctcaaaaaccaaagttatgaaacccggtccratcatcaacccgcttgargtagtggatcaatagtccaaccggtgggtcaataattcatctggtatatattaaaaaatttaaaattatatatgtatctattatatataagtatataactaacattatttgattaagaaaarttcattcatactccaaaatccaaaataacaattaaagtattaaagttaataacacaagtccacaaataataattcaataacacaattaca

SEQ ID NO: 50 - DNA, 401 bp, Artificial Sequence
  Design Sequence gmchr3v140563114_201
actcttaccaaaatatatattaaattttttaatagtaaatattttttatttttaaaaaaatatcrgattaaataattcgtatgatttatatcaaaattaattgtcacaaaatttattatttaaatttayatgcgcattaaatatacattagaaattttagtattatgtatagcaacattatttattttataacataattgactcattaactcttacgaaagaagttytttcgtaagaataaagggtaaaattggaaaaatgagaaaatactgggtgcaccaacaataatgctgggtgcacctagcaacacccaaacttgtaagaaagacttggacaaaaaaaaaattgcaaaccaagaccattagggcaaaatataaggctagcaaaaaaacaccttgacc

SEQ ID NO: 51 - DNA, 401 bp, Artificial Sequence
  Design Sequence gmchr3v140576895_201
agcccgacggattaaactragtttatccgagttgattgcataatcataattaatggaaactgagaagtaaaaaataagtaaattttaatgtgcataatttawtttttttgtaaaaagaaaagaaacagaaaaaactaactactacctgggaaacgcaactcctgctgcatcagcaagaagccgcaagttgagatcagtttttggtttatgttacccacccaataatgtgcataactgtataatagaatacagtttgcaaattttatttaaaaaaactagagtttttaatttgagtttaaatcttcaatatggacttatgttaaacataaggtaagagaattttattgtttataagtaataatggtagtgttttactcgagtgagattacatccttaagata

SEQ ID NO: 52 - DNA, 401 bp, Artificial Sequence
  Design Sequence gmchr3v140606905_201
cacaaatctaactaatataaaatatttaagtaaaraaaaggtaaggttgcctataattataaaaaattaaaaattaggtagtgaacaaggactcgrcacataaaaattgagagcatttatttctttttcttgacgccaaatagaagcgatagtaatgtttgtaagtattatatargctttattgtgacayaatgtcaatatggtgtcgtggtgtagttggttatcacgtcagtctaacacactgaaggtctccggttcgagtccgggcgacgccattatattctataacatttaattttacacttctacacatatttttgggtcagaagctttacattataggctagcccacaagatggaaagagaaagcatgcaggcccaaatgaacaaaatgtaattac

SEQ ID NO: 53 - DNA, 618 bp, Artificial Sequence
  Design Sequence S04733-1
aatgaacttactatcataaaggttgagctaattttatatatatcancnntgcctctcccctagaattctcaaattgagagggttaaccaaaatgaatgtgcagttaactaactaactacaaggaaaatgccaaacagaagaaaataactaataaaagaggcattttcattttgaattagaagtataaaacagttcagtacatccagaacagaacgttttccaatggtatctaagttgttaaatgtggtcactattgtttttcttttaagggctggataggttcttcaaatcctcttcctttgcataccagcttggtattatttttgaaacatggggataaagatccttgtacgagttyctaatggttccttctgcaactcctgtggcaactgatatatctgcaccaacacactaggtcagatacaaatatgcaatagctcatacatgtgaatattcttttagaaacctatttgcaatcaaatttaactgcaaattcaaaattcaccacacaagaaatcagagagacgattttcatgaagaattaaaccaatcaaatatgtttgtatttcattgctttcatgtttcaccataagaagtgtggcacatggtcatagctgt

SEQ ID NO: 54 - DNA, 535 bp, Artificial Sequence
  Design Sequence S00145-1
aagaaaagggaggttgtggttgagaagactggtggcccggctgagagctatgacgattttgctgcatctttgccggagaatgattgcagatatgctgtctttgactatgatttcgaagaaaagggaggttgtggttgagaagactggtggcccggctgagagctatgacgattttgctgcatctttgccggagaatgattgcagatatgctgtctttgactatgatttcggcattgttgcaccaaccagcaagatgtttgtgaggcatgatacatgataggcattttcttgtgcaaaaagcaaaccctgtggcatatatgatcatatcatatgtttggcagtttcaaaataaccatgccatgcaattctmttaagcccatgtcaatattgcactgcttaagtgtcactctttgcttgttctctgattatggagctacatgttcattgtttgttgtcttggattaaatttcttgtcttattctttggacacattgtttttacatgattgggctgtcaatctcacctagtagaataa

SEQ ID NO: 55 - DNA, 401 bp, Artificial Sequence
  Design Sequence gmchr3v1400061269-201
caaagtccttgagaaattgttcatcactatttagagtctggcttgatgatratcagttaactggaaacatcgcagatgcatttggagtactcccagmtgcagaaataagttggttggtgagcyctcctgggagtggggtgaatgtgtgaacttaactcaaatggatatggaaagcaacaaactttctggtaaaattccatttgaggtaagtaagttgagtcaattagggcatctaagccwgcattccaatgaattctgtaayattccattttttataaattaatttaaaaagaattgttatttataaataaatagagttttagaaaaatgatgaggtttttgtaattaaataaataaggaaaaataactttattaaaataataatgatttgagagaaaata

SEQ ID NO: 56 - DNA, 401 bp, Artificial Sequence
  Design Sequence S12869-1
aagttgagggrgttaggggtsgaagtsatgatggtgcagtcttgggttaaggatgatggagtgtttgtggcggagatgagagccatggtgagggaaaatggtaacgggataaaggctagtgttatwgaagtgaaaaatgcccttaatcagatcataccccgtcatgaaccatacacacttgcttccagtgatcatttttarccatgcttttggactaaggtgagacggctgtgccaaacatgaaagatgtgtgttaaatgttaattgaataagtttgtgtaagaatttaactcattctatgtttggtcaaytaagtacgcgaattgatcatgtaaaatatcatggacttgcagggggtggagggttgtgccatacccatgattgtcctccttaattagcct

SEQ ID NO: 57 - DNA, 401 bp, Artificial Sequence
  Design Sequence gmchr3v140061119-201
gcattcccagagaatttggaaagagtaatccttctttgactcatgtctacctttcaaacagcttctctggagaactgcatcctgacttgtgcagtgatggtaagctagttattttggcagtcaataacaacagcttttcaggsccattgccaaagtccttgagaaattgttcatcactatttagagtctggcttgatgataatcagttaactggaaacatcgcagatgcatttggagtactcccagmtgcagaaataagttggttggtgagcyctcctgggagtggggtgaatgtgtgaacttaactcaaatggatatggaaagcaacaaactttctggtaaaattccatytgaggtaagtaagttgagtcaattagggcatctaagccwgcattccaatg

SEQ ID NO: 58 - DNA, 800 bp, Artificial Sequence
  Design Sequence S06578-1
gccaaaaaggtagcatataacttcattgaagttcatgccaaatagacctgaaaacgggtcccttcaaagttcacataagtgaacactagccagaaataagagtgtaacctttataaagaaatttttgtccccaggaattaggtaaggcattcaaagaaatccaatttgataacagctataatttggtttttgcttaaagtagcattgccaggaacataaatttagggtacaacaaagtgcaagatataattaaaccaaattaaatctgcaggttggatatgaactaatggtctcaatcaattctctcttcttccctttatatgtataaacaataaacattttttttttcaattttgaccaatatttccagttctatgtttacaatgttttgccaagctaasatgtcttccacaatttctaaatttttctgatctacattcacgacaaggttcaccactgcttttttgaataaaaagtggagcctcatatcacacatatccaaagttttcttagacccacatatctgcatcatgtcattattaatcctattgagtaccactgtccaaatgggatgacttgaccttagaggtgatctttgagtttcaaaggggtatttgtacaagaaacctcattcaacaatttatgtttccaaaccacctccccaaaccagagaagcaagagaaaacactgaaatgcaaaatgttcagcagaaaatgtatttttcttctcctttactcccttccctcatgattaaaaaaagtctttttgaggaatcatctgtggatggattgcaaattgac